(19) United States
(12) Patent Application Publication
(10) Pub. No.: US 2023/0310285 A1
(43) Pub. Date: Oct. 5, 2023

(54) DEVELOPER COMPOSITION COMPRISING DIRECT DYE

(71) Applicant: L'OREAL, Paris (FR)

(72) Inventors: Derek HENRY, Jackson Heights, NY (US); Michael DEGEORGE, Brick, NJ (US)

(21) Appl. No.: 18/186,996

(22) Filed: Mar. 21, 2023

Related U.S. Application Data
(60) Provisional application No. 63/325,229, filed on Mar. 30, 2022.

(30) Foreign Application Priority Data
Jun. 7, 2022 (FR) 2205423

(51) Int. Cl. A61K 8/22; A61K 8/34; A61K 8/06; A61Q 5/10

(52) U.S. Cl. CPC A61K 8/22; A61K 8/342; A61K 8/06; A61Q 5/10; A61K 2800/4324; A61K 2800/882

(57) ABSTRACT

The present disclosure relates to a developer composition comprising one or more direct dyes having at least one halogenated aromatic group, one or more oxidizing agents, and water; wherein the pH of the developer composition is from about 1 to about 4. The developer compositions are combined with hair coloring base compositions to form ready-to-use hair coloring compositions. Methods for making ready to use hair coloring compositions and methods for coloring hair are also disclosed.

### CROSS-REFERENCE TO RELATED APPLICATIONS

This application claims benefit of U.S. Ser. No. 63/325,229 filed Mar. 30, 2022, and benefit of French Application No. FR 2205423, filed on Jun. 7, 2022, which are incorporated herein by reference in their entirety.

### FIELD OF THE DISCLOSURE

The present disclosure relates to developer compositions comprising direct dyes, hair coloring base compositions, and ready-to-use hair coloring compositions. Methods for making the compositions and methods for coloring hair are also disclosed.

### SUMMARY

There are various types of hair coloring compositions for temporarily, semi-permanently, and permanently coloring hair. Most types of hair coloring compositions for permanently coloring hair use two parts. One part is a hair coloring base composition containing oxidative dye precursors (sometimes called “oxidative dyes”) and couplers, which interact with the oxidative dye precursors. The second part is a developer composition containing an oxidizing agent like hydrogen peroxide. The two parts are mixed immediately prior to use forming a ready-to-use hair coloring composition that is activated for coloring the hair.

The developer compositions of the instant disclosure include one or more direct dyes having at least one halogenated aromatic ring, preferably at least one halogenated aryl group, more preferably at least one halogenated phenyl group. The halogens are fluorine, chlorine, bromine, iodine, and astatine, occupying group VIIA of the periodic table. Additional direct dyes may optionally be included (or excluded) from the developer composition. The developer composition also includes one or more oxidizing agents, such as peroxides, and has a low pH, from about 1 to about 4 or 5.

The developer composition is combined with a hair coloring base composition to derive a ready-to-use hair coloring composition for application to hair. All types of hair can be treated (colored), including hair of the head, eye lashes, eyebrows, and body.

The pH of the developer composition is typically low, from about 1 to about 4 or 5, for example, from about 1 to about 4, about 1 to about 3, about 2 to about 4, or about 2 to about 3.

As noted above, the direct dyes include at least one halogenated aromatic ring. There are various classes of direct dyes that can include a halogenated aromatic ring, for example, triarylmethane dyes, xanthene dyes, azo dyes, etc., and mixtures thereof.

Oxidizing agents are compounds or elements that receive electrons originating from a different species in a redox reaction. An oxidant is a chemical compound which easily transfers atoms of oxygen or another substance to gain an electron. Oxidizing agents include hydrogen peroxide, inorganic alkali metal peroxides, organic peroxides, inorganic perhydrate salts, bromates, etc., and a mixture thereof. Hydrogen peroxide is a particularly useful oxidizing agent.

The developer compositions of the instant disclosure may optionally include one or more fatty alcohols, nonionic surfactants, and/or water-soluble organic solvents. In certain embodiments, the fatty acids have from 12 to 24 carbon atoms. Nonlimiting examples include cetyl alcohol, stearyl alcohol, cetearyl alcohol, behenyl alcohol, oleyl alcohol, lauryl alcohol, myristic or myristyl alcohol, arachidyl alcohol, lignoceryl alcohol, and mixtures thereof.

The developer compositions may optionally include one or more nonionic surfactants. Nonlimiting examples of nonionic surfactants include ethylene glycol ethers of fatty alcohols, fatty amides, polyoxyalkylene fatty amides, and mixtures thereof.

The developer compositions of the instant disclosure are aqueous. In addition to water, however, the developer compositions may optionally include one or more water-soluble organic solvents. Nonlimiting examples of water-soluble organic solvents include glycerin, mono-alcohols, polyols (polyhydric alcohols), glycols, and mixture thereof, e.g., glycerin, propylene glycol, butylene glycol, pentylene glycol, dipropylene glycol, hexylene glycol, ethanol, isopropanol, t-butyl alcohol, and mixture thereof.

Further to the above, in various embodiments, the developer composition includes:
(a) one or more direct dyes comprising a halogenated aromatic ring, preferably at least one halogenated aryl group, more preferably at least one halogenated phenyl group, chosen from triarylmethane dyes, xanthene dyes, azo dyes, and mixtures thereof;
(b) one or more oxidizing agents chosen from hydrogen peroxide, inorganic alkali metal peroxides, organic peroxides, inorganic perhydrate salts, bromates, and a mixture thereof;
(c) water;
(d) optionally, one or more fatty alcohols;
(e) optionally, one or more nonionic surfactants; and
(f) optionally, one or more water-soluble organic solvents chosen from glycerin, mono-alcohols, polyols (polyhydric alcohols), glycols, and a mixture thereof.

The developer composition is mixed with a hair coloring base composition to form a ready-to-use hair coloring composition. For example, the developer composition can be mixed with a hair coloring base composition in a weight ratio of about 5:1 to about 1:5, about 3:1 to about 1:3, or about 2:1 to about 1:2, or about 1:1.

The developer composition and the hair coloring base composition are separately contained prior to mixing. Thus, both compositions may be provided in a kit. Kits according to the instant disclosure include one or more developer compositions and one or more hair coloring base compositions, wherein the developer compositions and the hair coloring base compositions are separately contained. For example, the one or more developer compositions and the one or more hair coloring base compositions may be included in separate containers that are packaged together.

Methods for making a ready-to-use hair coloring compositions and methods for coloring hair with the compositions entail combining the developer composition with a hair coloring base composition to form a ready-to-use hair coloring composition. They can be combined, for example, in the ratios set forth above. The ready-to-use hair coloring composition is then applied to hair for a period of time (for processing), for example, for about 1 minute to about 1 hour, about 1 minutes to about 45 minutes, about 1 minute to about 30 minutes, about 1 minute to about 15 minutes, about 5 minutes to about 1 hour, about 5 minutes to about 45 minutes, or about 5 minutes to about 30 minutes. After the period of time has lapsed, the hair coloring composition may be rinsed or washed from the hair exposing the newly colored hair.

### DETAILED DESCRIPTION

Individuals often seek to permanently change the color of hair using oxidative hair coloring procedures, which typically rely on a combination of two parts, a hair coloring base composition, and a developer composition. Hair coloring base compositions often include oxidative dye precursors, couplers, and direct dyes, for imparting color to hair. The developer compositions include oxidizing agents, like hydrogen peroxide. The two parts are mixed immediately prior to application to the hair to form a ready-to-use hair coloring composition.

Oxidative dye precursors are typically colorless or weakly colored compounds, which, when combined with oxidizing agents, transition to provide colored species via a process of oxidative condensation. The shades obtained with oxidative dye precursors may be varied by combining them with one or more couplers. Couplers include, for example, aromatic meta-diamines, meta-aminophenols, meta-diphenols, and certain heterocyclic compounds, such as indole compounds.

The oxidizing agent(s) employed in permanent dyeing compositions may degrade the melanin of the hair, which, depending on the nature of the oxidizing agent, may lead to less pronounced lightening of the fibers. A common oxidizing agent used in hair lightening and coloring is hydrogen peroxide. Nonetheless, other oxidizing agents are also used, often in conjunction with hydrogen peroxide.

In general, hair coloring compositions are alkaline, having a high pH of about 9 and higher, and may generally require the presence of an alkalizing agent such as ammonia or an ammonia gas-generating compound and/or an amine or ammonium-based compound in amounts sufficient to achieve the desired alkalinity. The alkalizing agents help activate the oxidizing agents and cause the hair shaft to swell, thus allowing the small oxidative dye precursor molecules to penetrate the cuticle and cortex before the oxidation condensation process is completed. The resulting larger-sized colored complexes from the oxidative reaction are then trapped inside the hair fiber, permanently altering the color of the hair.

As already noted, direct dyes are usually included in the hair coloring base compositions, which have a pH of about 9 or higher. This is because the direct dyes are typically stable in a high pH environment. Stability is needed to ensure a sufficient shelf-life for hair coloring products (coloring base and developer compositions) because they are not always used immediately after manufacture. If the direct dyes chemically breakdown before use, they will not deposit the intended color to hair, i.e., they will not function properly. Furthermore, if the direct dyes cause stability problems to the compositions containing them, the compositions may phase-separate, suffer from undesirable color and/or odor changes, and sedimentation may result.

It has been challenging to successfully incorporate direct dyes into developer compositions without jeopardizing the integrity of the direct dyes and/or the stability of the developer composition. The low pH of developer compositions and the reactivity of oxidizing agents, such as hydrogen peroxide, tend to preclude their inclusion in developer compositions. Developer compositions have a low pH, especially compared to hair coloring base compositions. For example, developer compositions typically have a pH of about 1 to about 5, depending on the amount of hydrogen peroxide included in the composition and the existence of other alkaline components.

The inventors of the instant disclosure surprising discovered that certain direct dyes can successfully be included in developer compositions having a low pH. The direct dyes maintain their integrity and do not disrupt the stability of developer compositions. For example, the inventors discovered that direct dyes having at least one halogenated aromatic ring are surprisingly stable in developer compositions, do not disrupt the stability of the developer compositions, and provide robust color deposition to hair, which is long lasting.

Typically, hair coloring and bleaching procedures use preformulated hair coloring base compositions with standardized developers. This limits the coloring options to those provided by the hair coloring base composition. Incorporating direct dyes into the developer composition results in significantly more color-producing combinations. A particular “toning” developer composition (a developer composition according to the instant disclosure containing one or more direct dyes having a halogenated aromatic ring) can be combined with a variety of different hair coloring base compositions and influence the final color deposited to the hair, beyond what the hair coloring base composition would provide with a standardized developer composition (i.e., a developer composition without direct dyes). This allows for more control and choice when targeting a particular hair color.

In various embodiment, the developer compositions of the instant disclosure include:
(a) about 0.001 to about 5 wt. % of one or more direct dyes comprising a halogenated aromatic ring, preferably at least one halogenated aryl group, more preferably at least one halogenated phenyl group;
(b) about 1 to about 40 wt. % of one or more oxidizing agents; and
(c) about 60 to 90 wt. % of water;
   wherein the composition has a pH of from about 1 to about 4, and
   all weight percentages are based on the total weight of the composition.

Direct dyes comprising at least one halogenated aromatic ring may be chosen from all different classes of direct dyes, including, but not limited to xanthene dyes, triarylmethane dyes, azo dyes, etc., and mixtures thereof. Nonlimiting examples of xanthene dyes comprising at least one halogenated aromatic ring include D&C Red 28, D&C Red 27, Eosin Y, Eosin B, Erythrosine B, Rose Bengal, etc. Nonlimiting examples of triarylmethane dyes comprising at least one halogenated aromatic ring include Tetrabromophenol Blue, Tetrabromo-sulfonephthalein, Bromsulphthalein, Bromocresol Green, Bromothymol Blue, etc. Nonlimiting examples of azo dyes comprising at least one halogenated aromatic ring include Acid Red 337, Disperse Red 167, Basic Red 18, Disperse Red 118, etc. A more exhaustive but nonlimiting list of direct dyes comprising at least one halogenated aromatic ring is included later, under the heading “Direct Dyes Comprising A Halogenated Aromatic Ring.”

The total amount of the one or more direct dyes comprising at least one halogenated aromatic ring in the developer composition will vary. Nonetheless, in various embodiments, the total amount of the one or more direct dyes comprising at least one halogenated aromatic ring in the developer composition is from about 0.001 to about 5 wt. % based on the total weight of the developer composition. In further embodiments, the total amount of the one or more direct dyes comprising at least one halogenated aromatic ring in the developer composition is from about 0.001 to about 4 wt. %, about 0.001 to about 3 wt. %, about 0.001 to about 2 wt. %, about 0.001 to about 1 wt. %, about 0.001 to about 0.5 wt. %, about 0.005 to about 4 wt. %, about 0.005 to about 3 wt. %, about 0.005 to about 2 wt. %, about 0.005 to about 1 wt. %, about 0.005 to about 0.5 wt. %, about 0.01 to about 5 wt. %, about 0.01 to about 4 wt. %, about 0.01 to about 3 wt. %, about 0.01 to about 2 wt. %, about 0.01 to about 1 wt. %, about 0.01 to about 0.5 wt. %, based on the total weight of the composition.

Direct dyes generally other than specific direct dyes comprising a halogenated aromatic ring may optionally be included or excluded from the developer composition, provided that the developer composition includes a direct dye comprising at least one halogenated aromatic ring. Nonlimiting direct dyes generally that may optionally be included or excluded are set forth under the heading “Direct Dyes Generally.”

Nonlimiting examples of oxidizing agents include hydrogen peroxide, inorganic alkali metal peroxides, organic peroxides, inorganic perhydrate salts, bromates, and a mixture thereof. In certain embodiments, at least one oxidizing agent in the developer composition is hydrogen peroxide. In further embodiments, the developer composition includes hydrogen peroxide and optionally one or more further oxidizing agents, for example, one or more further oxidizing agents chosen from inorganic alkali metal peroxides, organic peroxides, inorganic perhydrate salts, bromates, and a mixture thereof. In further embodiments, the developer composition is free or essentially free of one or more (or all) oxidizing agent chosen from inorganic alkali metal peroxides, organic peroxides, inorganic perhydrate salts, bromates, and a mixture thereof.

The total amount of the one or more oxidizing agents in the developer composition will vary. Nonetheless, in various embodiments, the total amount of the one or more oxidizing agents in the developer composition is from about 1 to about 40 wt. %, based on the total weight of the developer composition. In further embodiments, the total amount of the one or more oxidizing agents in the developer composition is from about 1 to about 30 wt. %, about 1 to about 20 wt. %, about 1 to about 15 wt. %, about 2 to about 40 wt. %, about 2 to about 30 wt. %, about 2 to about 20 wt. %, about 2 to about 15 wt. %, about 5 to about 40 wt. %, about 5 to about 30 wt. %, about 5 to about 20 wt. %, about 5 to about 15 wt. %, about 7 to about 40 wt. %, about 7 to about 30 wt. %, about 7 to about 20 wt. %, about 7 to about 15 wt. %, about 8 to about 20 wt. %, or about 8 to about 15 wt. %, based on the total weight of the developer composition.

One or more fatty alcohols may optionally be included in the developer compositions. The term “fatty alcohol” means an alcohol comprising at least one hydroxyl group (OH), and comprising at least 8 carbon atoms, and which is neither oxyalkylenated (in particular neither oxyethylenated nor oxypropylenated) nor glycerolated. The fatty alcohols can be represented by: R—OH, wherein R denotes a saturated (alkyl) or unsaturated (alkenyl) group, linear or branched, optionally substituted with one or more hydroxyl groups, comprising from 8 to 40 carbon atoms, preferably 10 to 30 carbon atoms, more preferably 12 to 24 carbon atoms, and even more preferably 14 to 22 carbon atoms.

In some instances, the compositions include at least one solid fatty alcohol. It is preferable that the solid fatty alcohols are solid at ambient temperature and at atmospheric pressure (25° C., 780 mmHg), and are insoluble in water, that is to say they have a water solubility of less than 1% by weight, preferably less than 0.5% by weight, at 25° C., 1 atm.

The solid fatty alcohols may be represented by: R—OH, wherein R denotes a linear alkyl group, optionally substituted with one or more hydroxyl groups, comprising from 8 to 40 carbon atoms, preferably 10 to 30 carbon atoms, more preferably 12 to 24 carbon atoms, and even more preferably 14 to 22 carbon atoms.

In particular, it is possible to mention, alone or as a mixture: lauryl alcohol or lauryl alcohol (1-dodecanol); myristic or myristyl alcohol (1-tetradecanol); cetyl alcohol (1-hexadecanol); stearyl alcohol (1-octadecanol); arachidyl alcohol (1-eicosanol); behenyl alcohol (1-docosanol); lignoceryl alcohol (1-tetracosanol); ceryl alcohol (1-hexacosanol); montanyl alcohol (1-octacosanol); myricylic alcohol (1-triacontanol).

Preferably, the solid fatty alcohol is chosen from myristyl alcohol, cetyl alcohol, stearyl alcohol, myristyl alcohol, behenyl alcohol, olelyl alcohol, octyldodecanol, and mixtures thereof such as cetylstearyl or cetearyl alcohol.

The liquid fatty alcohols, in particular those containing C10-C34, preferably have branched carbon chains and/or have one or more, preferably 1 to 3 double bonds. They are preferably branched and/or unsaturated (C═C double bond) and contain from 12 to 40 carbon atoms.

The liquid fatty alcohols may be represented by: R—OH, wherein R denotes a C12-C24 branched or straight alkyl group or an alkenyl group, R being optionally substituted by one or more hydroxy groups. Preferably, the liquid fatty alcohol is a branched saturated alcohol. Preferably, R does not contain a hydroxyl group. These include oleic alcohol, linoleic alcohol, linolenic alcohol, isocetyl alcohol, isostearyl alcohol, 2-octyl-1-dodecanol, 2-butyloctanol, 2-hexyl-1-decanol, 2-decyl-1-tetradecanol, 2-tetradecyl-1-cetanol and mixtures thereof.

In some instances, the compositions include one or more fatty alcohols selected from decyl alcohol, undecyl alcohol, dodecyl alcohol, myristyl alcohol, cetyl alcohol, stearyl alcohol, cetearyl alcohol, isostearyl alcohol, isocetyl alcohol, behenyl alcohol, linalool, oleyl alcohol, myricyl alcohol and a mixture thereof.

The total amount of the one or more fatty alcohols in the developer composition, if present, will vary. Nonetheless, in various embodiments, the developer composition includes from about 0.1 to about 10 wt. % of the one or more fatty alcohols, based on the total weight of the developer composition. In further embodiment, the developer composition includes from about 0.1 to about 8 wt. %, about 0.1 to about 5 wt. %, about 0.5 to about 10 wt. %, about 0.5 to about 8 wt. %, about 0.5 to about 5 wt. %, about 1 to about 10 wt. %, about 1 to about 8 wt. %, about 1 to about 5 wt. %, about 2 to about 10 wt. %, about 2 to about 8 wt. %, or about 2 to about 5 wt. % of the one or more fatty alcohols, based on the total weight of the developer composition.

In various embodiments, the developer composition includes one or more surfactants, for example, one or more anionic surfactants, nonionic surfactants, amphoteric surfactants (zwitterionic surfactants), cationic surfactants and/or a mixture thereof. In a preferred embodiment, the hair coloring base composition includes at least one or more nonionic surfactants. In certain embodiments, the hair coloring base compositions is preferably free or essentially free from cationic surfactants. In another embodiment, the developer composition includes at least one nonionic surfactant and optionally one or more anionic surfactants.

The total amount of the one or more surfactants (all types of surfactants, e.g., nonionic, anionic, amphoteric/zwitterionic, cationic) in the developer composition will vary. Nonetheless, in various embodiments, the total amount of the one or more surfactants in the developer composition is from about 0.1 to about 10 wt. %, based on the total weight of the developer composition. In further embodiments, the total amount of the one or more surfactants in the developer compositions is from about 0.1 to about 8 wt. %, about 0.1 to about 6 wt. %, about 0.1 to about 5 wt. %, about 0.1 to about 3 wt. %, about 0.5 to about 10 wt. %, about 0.5 to about 8 wt. %, about 0.5 to about 6 wt. %, about 0.5 to about 5 wt. %, about 0.5 to about 3 wt. %, about 1 to about 10 wt. %, about 1 to about 8 wt. %, about 1 to about 6 wt. %, about 1 to about 5 wt. %, or about 1 to about 3 wt. %, based on the total weight of the developer composition.

In various embodiments, the hair developer composition includes one or more nonionic surfactants. Nonlimiting examples of nonionic surfactants include oxyethylenated amides, oxyethylenated fatty alcohols, and block-copolymer (polycondensate) surfactants of ethylene oxide and of propylene oxide, and a mixture thereof. In a preferred embodiment, the hair coloring base composition includes PEG-4 rapeseedamide (an oxyethylenated amide), deceth-3 (an oxyethylenated fatty alcohol), poloxamer 338 (block-copolymer (polycondensate) surfactants of ethylene oxide and of propylene oxide), or a combination thereof.

Nonlimiting examples of nonionic oxyethylenated amides are those of the following formula:

R—[(OCH2CH2)n—OCH2]ₚ—CO—N(R′)—(CH2CH2O)ₙ′H

in which:

p denotes 0 or 1,

n denotes a number ranging from 1 to 10 and preferably from 1 to 6,

n′ denotes a number ranging from 1 to 100 and preferably from 1 to 60,

R′ denotes a hydrogen atom or a CH2CH2OH radical and preferably a hydrogen atom, and

R denotes a C10-C30 and preferably C12-C22 alkyl or alkenyl radical.

Examples of these compounds include AMIDET A15 sold by the company Kao (INCI name: Trideceth-2 carboxamide MEA), ETHOMID HP 60 sold by the company Akzo Nobel (INCI name: PEG-50 Hydrogenated Palmamide) and AMIDET N sold by the company Kao (INCI name: PEG-4 Rapeseedamide).

In some cases, the developer compositions includes at least rapeseed amide oxyethylenated with 4 oxyethylene units (PEG-4 rapeseedamide).

Nonlimiting examples of fatty alcohols include saturated or unsaturated and linear or branched alcohols comprising from 6 to 30 carbon atoms and preferably from 8 to 30 carbon atoms, for instance, cetyl alcohol, isostearyl alcohol, stearyl alcohol and the mixture thereof (cetylstearyl alcohol), octyldodecanol, 2-butyloctanol, 2-hexyldecanol, 2-undecylpentadecanol, oleyl alcohol, linolenyl alcohol, ricinoleyl alcohol, undecylenyl alcohol and linoleyl alcohol, and mixtures thereof.

Nonlimiting examples of oxyethylenated fatty alcohols include those comprising less than 10 OE units, preferably chosen from oxyethylenated derivatives of saturated or unsaturated, linear or branched, preferably linear, C₈-C₃₀ and preferably C₁₂-C₂₂ fatty alcohols, for instance cetyl alcohol, oleyl alcohol, oleocetyl alcohol, lauryl alcohol, behenyl alcohol, cetearyl alcohol, stearyl alcohol and isostearyl alcohol, and mixtures thereof.

As oxyethylenated fatty alcohols comprising less than 10 OE units, mention may be made of oxyethylenated fatty alcohols comprising from 2 to 8 and preferably from 2 to 6 OE units, for instance products of addition of ethylene oxide and lauryl alcohol, for instance lauryl alcohol 2 OE (CTFA name: laureth-2), products of addition of ethylene oxide and stearyl alcohol, for instance stearyl alcohol 2 OE (CTFA name: steareth-2), products of addition of ethylene oxide and decyl alcohol, for instance decyl alcohol 3 OE (CTFA name: deceth-3), decyl alcohol 5 OE (CTFA name: deceth-5), products of addition of ethylene oxide and oleocetyl alcohol, for instance oleocetyl alcohol 5 OE (CTFA name: oleoceteth-5), and mixtures thereof. In some instances, deceth-3 may be particularly useful.

Furthermore, non-limiting examples of oxyethylenated fatty alcohols having an average degree of ethoxylation of 2 to 29 are, for example, laureth-2, oleth-2, ceteareth-2, laneth-2, laureth-3, oleth-3, ceteareth-3, laureth-4, oleth-4, ceteareth-4, laneth-4, laureth-5, oleth-5, ceteareth-5, laneth-5, deceth-4, deceth-7, laureth-7, oleth-7, coceth-7, ceteth-7, ceteareth-7, C11-15 pareth-7, laureth-9, oleth-9, ceteareth-9, laureth-10, oleth-10, beheneth-10, ceteareth-10, laureth-12, ceteareth-12, trideceth-12, ceteth-15, laneth-15, ceteareth-15, laneth-16, ceteth-16, oleth-16, steareth-16, oleth-20, ceteth-20, ceteareth-20, laneth-20, steareth-21, ceteareth-23, ceteareth-25, ceteareth-27, and a mixture thereof.

In some cases, the developer composition includes at least one nonionic surfactant chosen from oxyethylenated amides and/or at least one nonionic surfactant chosen from oxyethylenated (OE) fatty alcohol comprising less than 10 OE units, that may be chosen among those described above. In further embodiments, the developer composition includes at least one nonionic surfactant chosen from oxyethylenated amides and at least one nonionic surfactant chosen from oxyethylenated (OE) fatty alcohol comprising less than 10 OE units, that may be chosen among those described above.

Furthermore, the developer composition may include one or more nonionic surfactants that are block-copolymer (polycondensate) surfactants of ethylene oxide and of propylene oxide. The block-copolymer (polycondensate) surfactant of ethylene oxide and of propylene oxide may have a weight-average molecular weight ranging from 1000 to 20000, better from 1500 to 19000, from 2000 to 18000, or from 4000 to 17000.

Mention may be made, as block-copolymer (polycondensate) surfactant of ethylene oxide and of propylene oxide which may be used, of the polyethylene glycol/polypropylene glycol/polyethylene glycol triblock polycondensates sold under the “SYNPERONIC” names, such as “SYNPERONIC PE/F32” (INCI name: Poloxamer 108), “SYNPERONI. PE/F108” (INCI name: Poloxamer 338), “SYNPERONIC PE/L44” (INCI name: Poloxamer 124), “SYNPERONIC PE/L42” (INCI name: Poloxamer 122), “SYNPERONIC PE/F127” (INCI name: Poloxamer 407), “SYNPERONIC PE/F88” (INCI name: Poloxamer 238) or “SYNPERONIC PE/L64” (INCI name: Poloxamer 184), by Croda or also “LUTROL F68” (INCI name: Poloxamer 188), sold by BASF. In some instances, Poloxamer 338 may be particularly useful.

A more exhaustive but nonlimiting list of nonionic surfactants is provided later, under the heading “Nonionic Surfactants.”

The total amount of the one or more nonionic surfactants in the developer composition will vary. Nonetheless, in various embodiments, the total amount of the one or more nonionic surfactants in the developer composition is from about 0.1 to about 10 wt. %, based on the total weight of the developer composition. In further embodiments, the total amount of the one or more nonionic surfactants in the developer compositions is from about 0.1 to about 8 wt. %, about 0.1 to about 6 wt. %, about 0.1 to about 5 wt. %, about 0.1 to about 3 wt. %, about 0.5 to about 10 wt. %, about 0.5 to about 8 wt. %, about 0.5 to about 6 wt. %, about 0.5 to about 5 wt. %, about 0.5 to about 3 wt. %, about 1 to about 10 wt. %, about 1 to about 8 wt. %, about 1 to about 6 wt. %, about 1 to about 5 wt. %, or about 1 to about 3 wt. %, based on the total weight of the developer composition.

In various embodiments, the developer composition includes one or more water-soluble organic solvents (or simply “water-soluble solvents”). The term “water-soluble organic solvent” (and “water-soluble solvent”) is interchangeable with the term “water-miscible solvent” and means an organic compound that is liquid at 25° C. and at atmospheric pressure (760 mmHg), and it has a solubility of at least 50% in water under these conditions. In certain embodiments the one or more water-soluble organic solvents have a solubility of at least 60%, 70%, 80%, or 90% in water at 25° C. and at atmospheric pressure (760 mmHg). Non-limiting examples of water-soluble organic solvents include glycerin, alcohols (for example, C₁₋₃₀, C₁₋₁₅, C₁₋₁₀, or C₁₋₄ alcohols), polyols, glycols, and a mixture thereof. In certain embodiments, the one or more water-soluble organic solvents, if present, are chosen from alcohols such as ethyl alcohol, isopropyl alcohol, propyl alcohol, benzyl alcohol, and phenylethyl alcohol, or glycols or glycol ethers such as monomethyl, monoethyl and monobutyl ethers of ethylene glycol, propylene glycol or ethers thereof such as, for example, monomethyl ether of propylene glycol, butylene glycol, hexylene glycol, dipropylene glycol as well as alkyl ethers of diethylene glycol, for example monoethyl ether or monobutyl ether of diethylene glycol.

Further non-limiting but useful examples of water-soluble organic solvents include alkanediols (polyhydric alcohols) such as glycerin, 1,2,6-hexanetriol, trimethylolpropane, ethylene glycol, propylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, (caprylyl glycol), 1,2-hexanediol, 1,2-pentanediol, and 4-methyl-1,2-pentanediol; alkyl alcohols having 1 to 4 carbon atoms such as ethanol, methanol, butanol, propanol, and isopropanol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, and dipropylene glycol mono-iso-propyl ether; 2-pyrrolidone, N-methyl-2-pyrrolidone, 1,3-dimethyl-2-imidazolidinone, formamide, acetamide, dimethyl sulfoxide, sorbit, sorbitan, acetine, diacetine, triacetine, sulfolane, and a mixture thereof.

In various embodiments, the developer composition includes one or more water-soluble organic solvents chosen from glycols, C₁₋₄ alcohols, glycerin, and a mixture thereof; preferably the water-soluble organic solvent is chosen from glycerin, ethanol, isopropyl alcohol, dipropylene glycol, propylene glycol, hexylene glycol, caprylyl glycol, propylene glycol, and a mixture thereof.

In certain embodiments, the developer composition includes one or more polyhydric alcohols. Nonlimiting examples of polyhydric alcohols include glycerin, ethylene glycol, diethylene glycol, triethylene glycol, propylene glycol, dipropylene glycol, tripropylene glycol, 1,3-butanediol, 2,3-butanediol, 1,4-butanediol, 3-methyl-1,3-butanediol, 1,5-pentanediol, tetraethylene glycol, 1,6-hexanediol, 2-methyl-2,4-pentanediol, polyethylene glycol, 1,2,4-butanetriol, 1,2,6-hexanetriol, and a mixture thereof. Polyol compounds may also be used. Non-limiting examples include the aliphatic diols, such as 2-ethyl-2-methyl-1,3-propanediol, 3,3-dimethyl-1,2-butanediol, 2,2-diethyl-1,3-propanediol, 2-methyl-2-propyl-1,3-propanediol, 2,4-dimethyl-2,4-pentanediol, 2,5-dimethyl-2,5-hexanediol, 5-hexene-1,2-diol, and 2-ethyl-1,3-hexanediol, and a mixture thereof.

The total amount of the one or more water-soluble organic solvents in the developer composition will vary. Nonetheless, in various embodiments, the total amount of the one or more water-soluble organic solvents, if present, is from about 0.01 to about 30 wt. %, based on the total weight of the developer composition. In further embodiments, the total amount of the one or more water-soluble organic solvents in the developer composition is from about 0.01 to about 20 wt. %, about 0.01 to about 15 wt. %, about 0.01 to about 10 wt. %, about 0.01 to about 5 wt. %, about 0.01 to about 2 wt. %, about 0.05 to about 30 wt. %, about 0.05 to about 20 wt. %, about 0.05 to about 15 wt. %, about 0.05 to about 10 wt. %, about 0.05 to about 5 wt. %, about 0.05 to about 2 wt. %, about 0.1 to about 30 wt. %, about 0.1 to about 20 wt. %, about 0.1 to about 15 wt. %, about 0.1 to about 10 wt. %, about 0.1 to about 5 wt. %, about 0.1 to about 2 wt. %, about 0.5 to about 20 wt. %, about 0.5 to about 15 wt. %, about 0.5 to about 10 wt. %, about 0.5 to about 5 wt. %, or about 0.5 to about 2 wt. %, based on the total weight of the developer composition.

The amount of water in the developer compositions will vary. Nonetheless, in various embodiments, the developer composition includes water in an amount of about 50 to about 90 wt. %, based on the total weight of the developer composition. In further embodiments, the total amount of water in the developer composition is from about 55 wt. % to about 90 wt. %, about 60 to about 90 wt. %, about 65 to about 90 wt. %, about 70 to about 90 wt. %, about 75 to about 90 wt. %, about 80 to about 90 wt. %, about 50 to about 86 wt. %, about 55 to about 86 wt. %, about 60 to about 86 wt. %, about 65 to about 86 wt. %, about 70 to about 86 wt. %, about 75 to about 86 wt. %, or about 80 to about 86 wt. %, based on the total weight of the developer composition.

In various embodiments, the developer composition includes one or more fatty compounds that are not fatty alcohols, preferably one or more non-silicone fatty compounds. The term “non-silicone fatty compound” means a fatty compound that does not containing any silicon atoms (Si). Non-limiting examples of non-silicone fatty compounds include oils, mineral oil, fatty acids, fatty alcohol derivatives, fatty acid derivatives (such as alkoxylated fatty acids or polyethylene glycol esters of fatty acids or propylene glycol esters of fatty acids or butylene glycol esters of fatty acids or esters of neopentyl glycol and fatty acids or polyglycerol/glycerol esters of fatty acids or glycol diesters or diesters of ethylene glycol and fatty acids or esters of fatty acids and fatty alcohols, esters of short chain alcohols and fatty acids), esters of fatty alcohols, hydroxy-substituted fatty acids, waxes, triglyceride compounds, lanolin, and a mixture thereof. Non-limiting examples of fatty acids, fatty alcohol derivatives, and fatty acid derivatives are found in International Cosmetic Ingredient Dictionary, Sixteenth Edition, 2016, which is incorporated by reference herein in its entirety. A more exhaustive but nonlimiting list of fatty compounds is provided later, under the heading “Fatty Compounds Other Than Fatty Alcohols.”

In various embodiments, the one or more fatty compounds are liquid fatty compounds, also referred to as “oils.” The term “oil” or “liquid fatty compound” as used herein refers to an organic compound insoluble in water at normal temperature (25° C.) and at atmospheric pressure (760 mmHg), i.e. it has a water solubility of less than 5% by weight, or less than 1% by weight, or less than 0.1% by weight. Furthermore, oils are generally soluble in organic solvents in the same conditions of temperature and pressure, for example in chloroform, ethanol, benzene or decamethylcyclopentasiloxane. Furthermore, oils are liquid at ordinary temperature (25° C.) and at atmospheric pressure (760 mmHg).

Fatty compounds and oils are useful when the developer composition is in the form of an emulsion. The developer composition may in the form of an oil in water emulsion or a water in oil emulsion.

The total amount of the one or more fatty compounds other than fatty alcohols, preferably one or more oils, will vary. Nonetheless, in various embodiments, the total amount of the one or more fatty compounds, preferably one or more oils is from about 1 to about 1 to about 40 wt. %, based on the total weight of the developer composition. In further embodiments, the total amount of the one or more fatty compounds, preferably one or more oils, is from about 1 to about 30 wt. %, about 1 to about 25 wt. %, about 4 to about 40 wt. %, about 5 to about 30 wt. %, about 5 to about 25 wt. %, about 10 to about 40 wt. %, about 10 to about 35 wt. %, about 10 to about 30 wt. %, or about 10 to about 25 wt. %, based on the total weight of the developer composition.

The developer composition may optionally include one or more conditioning agents. For example, the developer compositions may optionally include one or more glyceryl ethers as conditioning agent(s). Non-limiting examples of glyceryl ethers include glyceryl butyl ether, glyceryl isobutyl ether, glyceryl tert-butyl ether, glyceryl pentyl ether, glyceryl isopentyl ether, glyceryl hexyl ether, glyceryl isohexyl ether, glyceryl heptyl ether, glyceryl octyl ether, glyceryl ethylhexyl ether, glyceryl nonyl ether, glyceryl decyl ether, glyceryl isodecyl ether, glyceryl lauryl ether, glyceryl myristyl ether, glyceryl palmityl ether, glyceryl stearyl ether and glyceryl behenyl ether and their mixtures. Particularly useful glyceryl ethers also include glyceryl butyl ether, glyceryl isobutyl ether, glyceryl tert-butyl ether, glyceryl pentyl ether, glyceryl isopentyl ether, glyceryl hexyl ether, glyceryl isohexyl ether, glyceryl heptyl ether, glyceryl octyl ether, glyceryl ethylhexyl ether, glyceryl nonyl ether, glyceryl decyl ether, glyceryl isodecyl ether, glyceryl lauryl ether, and a mixture thereof. In some instances, glyceryl lauryl ether is particularly useful.

The total amount of the one or more conditioning agents in the developer composition may vary but if present, is typically in an amount of about 0.1 to about 15 wt. %, based on the total weight of the developer composition. In various embodiments, the total amount of the one or more conditioning agents in the developer composition is from about 0.1 to about 10 wt. %, 0.1 to about 5 wt. %, about 0.5 to about 15 wt. %, about 0.5 to about 10 wt. %, about 0.5 to about 5 wt. %, or about 1 to about 10 wt. %, or about 1 to about 5 wt. %, based on the total weight of the developer composition.

In various embodiments, the developer composition includes one or more cationic conditioning polymers. Nonlimiting examples include cationic polysaccharides derivatives, cationic gum derivatives, polymer derivatives of diallyldimethyl ammonium chloride, polymer derivatives of methacrylamidopropyltrimethylammonium chloride, cationic cellulose derivatives, quaternized hydroxyethyl cellulose, cationic starch derivatives, cationic guar gum derivatives (hydroxypropyl guar hydroxypropyltrimonium chloride), copolymers of acrylamide and dimethyldiallyammonium chloride, polyquaterniums, and a mixture thereof. A more exhaustive but nonlimiting list of cationic conditioning polymers is included later, under the heading “Cationic Conditioning Polymers.”

The total amount of the one or more cationic conditioning polymers, if present, will vary. Nonetheless, in various embodiments, the total amount of the one or more cationic conditioning polymers in the developer composition is from about 0.01 to about 10 wt. %, about 0.01 to about 5 wt. %, about 0.01 to about 3 wt. %, about 0.1 to about 10 wt. %, about 0.1 to about 5 wt. %, or about 0.1 to about 3 wt. %, based on the total weight of the developer composition.

In various embodiments, the developer composition includes one or more thickening agents. Nonlimiting examples of thickening agents include polyacrylate crosspolymers or crosslinked polyacrylate polymers, cationic acrylate copolymers, anionic acrylic or carboxylic acid polymers, polyacrylamide polymers, polysaccharides, gums, polyquaterniums, vinylpyrrolidone homopolymers/copolymers, C8-24 hydroxyl substituted aliphatic acid, C8-24 conjugated aliphatic acid, sugar fatty esters, polyglyceryl esters, and a mixture thereof. A more exhaustive but non-limiting list of thickening agents is included later, under the heading “Thickening Agents.”

The total amount of the one or more thickening agents, if present, will vary. For example, in various embodiments, the developer composition includes one or more thickening agents in an amount of about 0.01 to about 10 wt. %, about 0.01 to about 8 wt. %, about 0.01 to about 5 wt. %, about 0.01 to about 3 wt. %, about 0.1 to about 10 wt. %, about 0.1 to about 8 wt. %, about 0.1 to about 5 wt. %, about 0.1 to about 3 wt. %, based on the total weight of the developer composition.

The hair coloring compositions of the instant disclosure (developer compositions, hair coloring base compositions, and/or the ready-to-use hair coloring compositions) may optionally include (or optionally exclude) one more miscellaneous ingredients. Miscellaneous ingredients are ingredients that are compatible with the developer composition and/or hair coloring base composition but do not disrupt or materially affect the basic and novel properties of the compositions. Nonlimiting examples of ingredients include preservatives, fragrances, pH adjusters, salts, chelating agents, buffers, antioxidants, flavonoids, vitamins, botanical extracts, UV filtering agents, proteins, protein hydrolysates, and/or isolates, fillers, composition colorants, cationic polymers, thickening agents, etc. In various embodiments, the miscellaneous ingredients are chosen from preservatives, fragrances, pH adjusters, salts, chelating agents, buffers, composition colorants, and mixtures thereof. In the context of the instant disclosure, a “composition colorant” is a compound that colors the composition but does not have an appreciable coloring effect on hair. In other words, the composition colorant is included to provide a coloring to the composition for aesthetic appeal but is not intended to impart coloring properties to hair. Styling gels, for example, can be found in a variety of different colors (e.g., light blue, light pink, etc.) yet application of the styling gel to hair does not visibly change the color of the hair.

The total amount of the one or more miscellaneous ingredients, if present, will vary. Nonetheless, in various embodiments, the compositions of the instant disclosure (developer compositions, hair coloring base compositions, and/or the ready-to-use hair coloring compositions) include, if present, from about 0.001 to about 10 wt. % of one or more miscellaneous ingredients, based on the total weight of the composition. In further embodiments, the compositions of the instant disclosure include from about 0.001 to about 5 wt. %, about 0.001 to about 3 wt. %, about 0.01 to about 10 wt. %, about 0.01 to about 5 wt. %, about 0.01 to about 3 wt. %, about 0.1 to about 10 wt. %, about 0.1 to about 5 wt. %, or about 0.1 to about 3 wt. % of one or more miscellaneous ingredients, based on the total weight of the composition.

In various embodiments, the developer composition is free or essentially free from reducing agents. Reducing agents are chemical species that lose an electron to another chemical species in a redox chemical reaction. Reducing agents are typically include in the hair coloring base compositions. Nonlimiting examples of reducing agents include ammonium bisulfite, ammonium sulfite, potassium metabisulfite, potassium sulfite, sodium hydrosulfite, sodium metabisulfite, sodium sulfite, sodium bisulphite, thioglycolic acid, thiolactic acid, dehydroascorbic acid, a salt thereof, and a mixture thereof.

In various embodiments, the developer composition is free or essentially free from oxidative dye precursors. Oxidative dye precursors are generally included in the hair coloring base compositions. Oxidative dye precursors are typically colorless or weakly colored compounds, which, when combined with oxidizing agents, transition to provide colored species via a process of oxidative condensation. The shades obtained with oxidative dye precursors may be varied by combining them with one or more couplers. Nonlimiting examples of couplers include aromatic meta-diamines, meta-aminophenols, meta-diphenols, and certain heterocyclic compounds, such as indole compounds.

In various embodiments, the developer compositions of the instant disclosure comprises or consists of:
(a) about 0.001 to about 4 wt. %, preferably about 0.005 to about 3 wt. %, more preferably about 0.01 to about 1 wt. % of one or more direct dyes comprising at least one halogenated aromatic ring, preferably at least one halogenated aryl group, more preferably at least one halogenated phenyl group, chosen from triarylmethane dyes, xanthene dyes, azo dyes, and mixtures thereof, wherein:
   the triarylmethane dyes are chosen from tetrabromophenol blue, 3,4,5,6-tribromophenol sulfone phthalein, sulfobromophthalein, bromocresol green, bromothymol blue, and mixtures thereof;
   D&C Red 28, Erythrosine B, Eosin Y, Eosin B, (eosin (2′,4′,5′,7′-tetrabromo-fluorescein) methyl ester, eosin (2′,4′,5′,7′-tetrabromo-fluorescein) p-isopropylbenzyl ester, eosin derivative (2′,7′-dibromo-fluorescein), eosin derivative (4′,5′-dibromo-fluorescein), eosin derivative (2′,7′-dichloro-fluorescein), eosin derivative (4′,5′-dichloro-fluorescein), eosin derivative (2′,7′-diiodo-fluorescein), eosin derivative (4′,5′-diiodo-fluorescein), eosin derivative (tribromo-fluorescein), eosin derivative (2′,4′,5′,7′-tetrachloro-fluorescein), erythrosin B (2′,4′,5′,7′-tetraiodo-fluorescein), erythiosin B; fluorescein; fluorescein dianion, phloxin B (2′,4′,5′,7′-tetrabromo-3,4,5,6-tetrachloro-fluorescein, dianion), phloxin B (tetrachloro-tetrabromo-fluorescein), phloxine B, rose bengal (3,4,5,6-tetrachloro-2′,4′,5′,7′-tetraiodofluorescein, dianion), pyronin G, pyronin J, pyronin Y, 4,5-dibromo-rhodamine methyl ester, 4,5-dibromo-rhodamine n-butyl ester, rhodamine 101 methyl ester, rhodamine 123, rhodamine 6G, rhodamine 6G hexyl ester, tetrabromo-rhodamine 123, tetramethyl-rhodamine ethyl ester, and mixtures thereof; and
   the azo dyes are chosen from Acid Red 337, Disperse Red 167, Basic Red 18, Disperse Red 118, and mixtures thereof;
(b) about 1 to about 40 wt. %, preferably about 2 to about 30 wt. %, more preferably about 5 to about 20 wt. % of hydrogen peroxide, and optionally one or more further oxidizing agents, for example, one or more further oxidizing agents chosen from inorganic alkali metal peroxides, organic peroxides, inorganic perhydrate salts, bromates, and a mixture thereof'
(c) about 50 to 90 wt. %, preferably about 70 to about 90 wt. %, more preferably about 75 to about 86 wt. % of water;
(d) about 0.1 to about 10 wt. %, preferably about 0.5 to about 8 wt. %, more preferably about 1 to about 6 wt. % of one or more fatty alcohols having from 12 to 24 carbon atoms, preferably chosen from decyl alcohol, undecyl alcohol, dodecyl alcohol, myristyl alcohol, cetyl alcohol, stearyl alcohol, cetearyl alcohol, isostearyl alcohol, isocetyl alcohol, behenyl alcohol, linalool, oleyl alcohol, myricyl alcohol and a mixture thereof, more preferably, cetearyl alcohol;
(e) about 0.1 to about 10 wt. %, preferably about 0.5 to about 8 wt. %, more preferably about 1 to about 5 wt. % of one or more nonionic surfactants, preferably one or more ethylene glycol ethers of fatty alcohols, fatty amides, polyoxyalkylene fatty amides, and mixtures thereof, more preferably chosen from ceteareth-25, steareth-20, PEG-4 rapeseedamine, and mixtures thereof;
(f) about 0.01 to about 30 wt. %, preferably about 0.1 to about 20 wt. %, more preferably about 0.2 to about 10 wt. % of one or more water-soluble organic solvents chosen from glycerin, mono-alcohols, polyols (polyhydric alcohols), glycols, and a mixture thereof, preferably one or more water-soluble organic solvent chosen from glycerin, ethanol, isopropyl alcohol, dipropylene glycol, propylene glycol, hexylene glycol, caprylyl glycol, propylene glycol, and a mixture thereof, more preferably glycerin;
(g) optionally, about 0.01 to about 6 wt. %, about 0.1 to about 5 wt. %, more preferably about 0.5 to about 4 wt. % of one or more miscellaneous ingredients, for example, one or more miscellaneous ingredients chosen from fragrances, preservatives, chelating agents, pH adjusters, buffering agents, botanical extracts, and a mixture thereof.

wherein the developer composition has a pH of from about 1 to about 5, about 1 to about 4, or about 1 to about 3, and all weight percentages are based on the total weight of the developer composition.

In various embodiments, the developer compositions of the instant disclosure is in the form of an emulsion, preferably an oil in water emulsion, and comprises or consists of:
(a) about 0.001 to about 4 wt. %, preferably about 0.005 to about 3 wt. %, more preferably about 0.01 to about 1 wt. % of one or more direct dyes comprising at least one halogenated aromatic ring, preferably at least one halogenated aryl group, more preferably at least one halogenated phenyl group, chosen from triarylmethane dyes, xanthene dyes, azo dyes, and mixtures thereof, wherein:
   the triarylmethane dyes are chosen from tetrabromophenol blue, 3,4,5,6-tribromophenol sulfone phthalein, sulfobromophthalein, bromocresol green, bromothymol blue, and mixtures thereof;
   the xanethene dyes are chosen D&C Red 28, Erythrosine B, Eosin Y, Eosin B, (eosin (2′,4′,5′,7′-tetrabromo-fluorescein) methyl ester, eosin (2′,4′,5′,7′-tetrabromo-fluorescein) p-isopropylbenzyl ester, eosin derivative (2′,7′-dibromo-fluorescein), eosin derivative (4′,5′-dibromo-fluorescein), eosin derivative (2′,7′-dichloro-fluorescein), eosin derivative (4′,5′-dichloro-fluorescein), eosin derivative (2′,7′-diiodo-fluorescein), eosin derivative (4′,5′-diiodo-fluorescein), eosin derivative (tribromo-fluorescein), eosin derivative (2′,4′,5′,7′-tetrachloro-fluorescein), erythrosin B (2′,4′,5′,7′-tetraiodo-fluorescein), erythiosin B; fluorescein; fluorescein dianion, phloxin B (2′,4′,5′,7′-tetrabromo-3,4,5,6-tetrachloro-fluorescein, dianion), phloxin B (tetrachloro-tetrabromo-fluorescein), phloxine B, rose bengal (3,4,5,6-tetrachloro-2′,4′,5′,7′-tetraiodofluorescein, dianion), pyronin G, pyronin J, pyronin Y, 4,5-dibromo-rhodamine methyl ester, 4,5-dibromo-rhodamine n-butyl ester, rhodamine 101 methyl ester, rhodamine 123, rhodamine 6G, rhodamine 6G hexyl ester, tetrabromo-rhodamine 123, tetramethyl-rhodamine ethyl ester, and mixtures thereof; and
   the azo dyes are chosen from Acid Red 337, Disperse Red 167, Basic Red 18, Disperse Red 118, and mixtures thereof;
(b) about 1 to about 40 wt. %, preferably about 2 to about 30 wt. %, more preferably about 5 to about 20 wt. % of hydrogen peroxide, and optionally one or more further oxidizing agents, for example, one or more further oxidizing agents chosen from inorganic alkali metal peroxides, organic peroxides, inorganic perhydrate salts, bromates, and a mixture thereof'
(c) about 50 to 90 wt. %, preferably about 70 to about 90 wt. %, more preferably about 75 to about 86 wt. % of water;
(d) about 0.1 to about 10 wt. %, preferably about 0.5 to about 8 wt. %, more preferably about 1 to about 6 wt. % of one or more fatty alcohols having from 12 to 24 carbon atoms, preferably chosen from decyl alcohol, undecyl alcohol, dodecyl alcohol, myristyl alcohol, cetyl alcohol, stearyl alcohol, cetearyl alcohol, isostearyl alcohol, isocetyl alcohol, behenyl alcohol, linalool, oleyl alcohol, myricyl alcohol and a mixture thereof, more preferably, cetearyl alcohol;
(e) about 0.1 to about 10 wt. %, preferably about 0.5 to about 8 wt. %, more preferably about 1 to about 5 wt. % of one or more nonionic surfactants, preferably one or more ethylene glycol ethers of fatty alcohols, fatty amides, polyoxyalkylene fatty amides, and mixtures thereof, more preferably chosen from ceteareth-25, steareth-20, PEG-4 rapeseedamine, and mixtures thereof;
(f) about 0.01 to about 30 wt. %, preferably about 0.1 to about 20 wt. %, more preferably about 0.2 to about 10 wt. % of one or more water-soluble organic solvents chosen from glycerin, mono-alcohols, polyols (polyhydric alcohols), glycols, and a mixture thereof, preferably one or more water-soluble organic solvent chosen from glycerin, ethanol, isopropyl alcohol, dipropylene glycol, propylene glycol, hexylene glycol, caprylyl glycol, propylene glycol, and a mixture thereof, more preferably glycerin;
(g) about 1 to about 40 wt. %, preferably about 1 to about 30 wt. %, more preferably about 5 to about 25 wt. % of one or more fatty compounds other than the one or more fatty alcohols, preferably one or more non-silicone oils, for example, mineral oil;
(h) optionally, about 0.01 to about 6 wt. %, about 0.1 to about 5 wt. %, more preferably about 0.5 to about 4 wt. % of one or more miscellaneous ingredients, for example, one or more miscellaneous ingredients chosen from fragrances, preservatives, chelating agents, pH adjusters, buffering agents, botanical extracts, and a mixture thereof.

wherein the developer composition has a pH of from about 1 to about 5, about 1 to about 4, or about 1 to about 3, and all weight percentages are based on the total weight of the developer composition.

The developer compositions and the direct dyes comprising at least one halogenated aromatic ring are surprisingly stable and effective. Stability can be assessed, for example, by the longevity (maintenance) of pH, peroxide content, and/or viscosity.

In various embodiments, the pH of the developer composition does not change by more than 15%, preferably 10%, in storage at a temperature of 25° C., 4° C., 37° C., or 45° C. for at least 1 month, preferably at least 2 months. Furthermore, in certain embodiments, the pH of the developer composition does not change by more 15%, preferably 10%, in storage at a temperature of 25° C. for at least 6 months.

In various embodiments, the amount of hydrogen peroxide in the developer compositions does not change by more than 10%, 5%, or 2% after 1 week in storage at 25° C. In further embodiments, the amount of hydrogen peroxide in the developer compositions does not change by more than 10%, 5%, or 2% after 2 weeks of storage, 1 month of storage 2 months of storage, 3 months of storage, 6 months of storage, or 1 year of storage at 25° C. The hydrogen peroxide content can be measured, for example, by titration with Potassium Permanganate, using standard techniques. For example, as described by J. S. Reichert (Determination of Hydrogen Peroxide and Some Related Peroxygen Compounds, IND. ENG. CHEM. ANAL. ED. 1939, 11, 4, 194-197).

In various embodiment, the viscosity of the developer composition does not change by more than 15%, preferably 10%, more preferably 5%, in storage at a temperature of 25° C., 4° C., 37° C., or 45° C. for at least 1 month, preferably at least 2 months. Furthermore, in certain embodiments, the viscosity of the developer composition does not change by more than 15%, preferably 10%, more preferably 5%, in storage at a temperature of 25° C. for at least 6 months. The viscosity measurements can be carried out, for example, using a Brooksfield viscometer/rheometer with a #5 spindle at 12 RPM, using a Brooksfield viscometer.

The developer compositions of the instant disclosure may be included in a kit. For example, a kit comprising one or more developer compositions according to the instant disclosure and one or more hair coloring base compositions, wherein the one or more developer compositions and the one or more hair coloring base compositions are separately contained. For example, the one or more developer compositions and the one or more hair coloring base compositions may be included in separate containers that are packed together or included in different compartments of a single container or device, for example, a device that combines the developer composition with a hair coloring base composition.

The instant disclosure relates to methods for making a ready-to-use hair coloring compositions and to methods for coloring hair. In various embodiments, the methods include combining a developer composition of the instant disclosure with a hair coloring base composition to form a ready-to-use hair coloring composition. The ready-to-use hair coloring composition is applied to hair for a period of time (for processing), for example, for about 1 minute to about 1 hour, about 1 minute to about 45 minutes, about 1 minute to about 30 minutes, about 2 minutes to about 1 hour, about 2 minutes to about 45 minutes, about 2 minutes to about 30 minutes, about 5 minutes to about 1 hour, about 5 minutes to about 45 minutes, about 5 minutes to about 30 minutes, or about 5 minutes to about 20 minutes. After the period of time has lapsed, the hair coloring compositions may be rinsed or washed from the hair exposing the newly colored hair.

The developer compositions of the instant disclosure can be in a variety of different forms. For example, the developer composition may be in the form of a liquid, gel, foam, lotion, cream, mousse, emulsion, etc. In various embodiments, the developer composition is in the form of an emulsion. In other various embodiments, the developer is in the form of an emulsion, for example, an oil in water emulsion.

The viscosity of the developer composition will vary depending on the form of the composition, etc. Nonetheless, I various embodiments, the viscosity of the developer composition is from about 1 cPs to about 25,000 cPs at 25° C. using a #5 spindle at 12 RPM, using a Brooksfield viscometer. In further embodiments, the viscosity of the developer composition is from about 1 to about 20,000 cPs, about 1 to about 15,000 cPs, about 1 to about 10,000 cPs, about 1 to about 5,000 cPs, about 1 to about 2,000 cPs, or about 1 to about 1,000 cPs. In further embodiments, the viscosity is from about 1,000 to about 25,000 cPs, about 2,000 to about 25,000 cPs, about 5,000 to about 25,000 cPs, about 10,000 to about 25,000 cPs, about 15,000 to about 25,000 cPs, or about 10,000 to about 20,000 cPs at 25° C. using a #5 spindle at 12 RPM, using a Brooksfield viscometer.

Nonlimiting examples of direct dyes comprising at least one halogenated aromatic rings are shown in the following tables.

Halogenated Xanthene Dyes

| Dye | Synonym | CAS | Chemical Name |
|---|---|---|---|
| D&C Red 28 | Acid Red 92, | 18472-87-2 | 3,4,5,6-tetrachloro-2-(1,4,5,8-tetrabromo-6- |
| | Phloxine B | | hydroxy-3-oxoxanthen-9-yl)benzoic acid |
| D&C Red 27 | Phloxine O | 13473-26-2 | 2′,4′,5′,7′-tetrabromo-4,5,6,7-tetrachloro-3′,6′- |
| | | | dihydroxyspiro[2-benzofuran-3,9′-xanthene]-1-on |
| Eosin Y | Acid Red 87 | 17372-87-1 | 2-(2,4,5,7-tetrabromo-6-oxido-3-oxo-3H-xanthen- |
| | | | 9-yl)benzoate |
| Eosin B | Acid Red 91 | 548-24-3 | 4′,5′-dibromo-3′,6′-dihydroxy-2′,7′-dinitro-1- |
| | | | spiro[isobenzofuran-3,9′-xanthene]on |
| Erythrosine B | Red No. 3, | 16423-68-0 | 2-(6-hydroxy-2,4,5,7-tetraiodo-3-oxo-xanthen-9- |
| | Acid Red 51 | | yl)benzoic acid |
| Rose Bengal | Acid Red 94 | 4159-77-7 | 4,5,6,7-Tetrachloro-3′,6′-dihydroxy-2′,4′,5′,7′- |
| | | | tetraiodo-3H-spiro[[2]benzofuran-1,9′-xanthen]-3- |
| | | | one |
| Tetrabromophenol | | 4430-25-5 | 3,3-Bis(3,5-dibromo-4-hydroxyphenyl)-2,1λ6- |
| Blue | | | benzoxathiole-1,1(3H)-dione |
| Tetrabromo- | | 77172-72-6 | 3,4,5,6-tetrabromophenol-sulfonephthalein |
| sulfonephthalein | | | |
| Bromsulphthalein | Sulfobromo- | 71-67-0 | Disodium 3,3′-(4,5,6,7-tetrabromo-3-oxo-2- |
| | phthalein | | benzofuran-1,1(3H)-diyl)bis(6-hydroxybenzene- |
| | | | 1-sulfonate) |
| Bromocresol | BCG | 76-60-8 | 3,3-Bis(3,5-dibromo-4-hydroxy-2-methylphenyl)- |
| Green | | | 2,1λ6-benzoxathiole-1,1(3H)-dione |
| Bromothymol Blue | Bromothymol | 76-59-5 | 3,3-Bis[3-bromo-4-hydroxy-2-methyl-5-(propan- |
| | sulfone | | 2-yl)phenyl]-2,1λ6-benzoxathiole-1,1(3H)-dione |
| | phthalein | | |
| Acid Red 337 | | 67786-14-5 | sodium;6-amino-4-hydroxy-5-[[2- |
| | | | (trifluoromethyl)phenyl]diazenyl]naphthalene-2- |
| | | | sulfonate |
| Disperse Red | | 61968-52-3 | 2′-Chloro-4′-nitro-4-[bis(2-acetoxyethyl)amino]-2- |
| 167 | | | (acetylamino)azobenzene; 2′-(Acetylamino)-4′- |
| | | | [bis(2-acetoxyethyl)amino]-2-chloro-4- |
| | | | nitroazobenzene |
| Basic Red 18 | Astrazon Red | 14097-03-1 | 2-[{4-[(E)-(2-chloro-4- |
| | GTL | | nitrophenyl)diazenyl]phenyl}(ethyl)amino]-N,N,N- |
| | | | trimethylethanaminium |
| Disperse Red | Palanil Brown | 52623-75-3 | 2,2′-[[3-chloro-4-[(2,6-dibromo-4- |
| 118 | 3REL | | nitrophenyl)azo]phenyl]imino]bisethanol |
| HC Yellow 16 | | 1184721-10- | 2-chloro-4-[(1E)-(1-methyl-1H-pyrazol-5- |
| | | 5 | yl)diazenyl]-phenol |
| HC Blue 18 | | 1166834-57- | 3-[(E)-(3-chloro-4-hydroxyphenyl)diazenyl]-2,l- |
| | | 6 | benzisothiazole-5- sulphonamide |
| | | | 2-chloro-4-[(E)-(3-methyl-1,2,4-thiadiazol-5- |
| | | | yl)diazenyl]phenol |

Nonlimiting examples of aromatic rings that can be halogenated and form part of the overall structure of the direct dye are shown below (without halogenation).

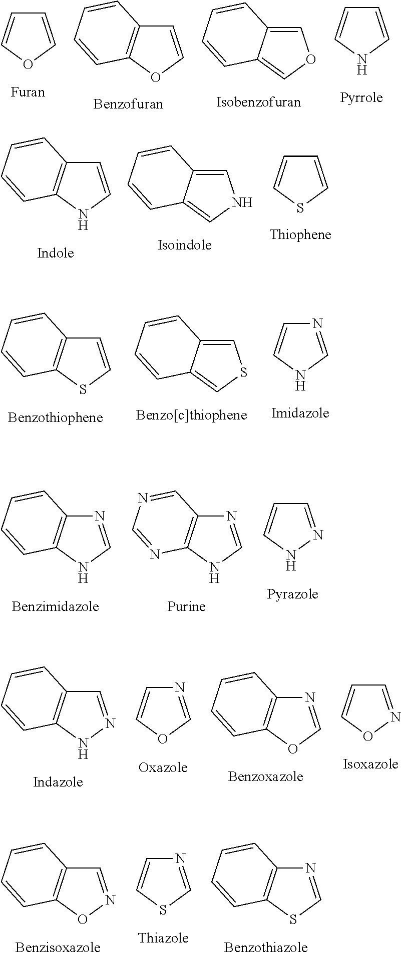

### Six-Membered Rings and Fused Six-Membered Rings

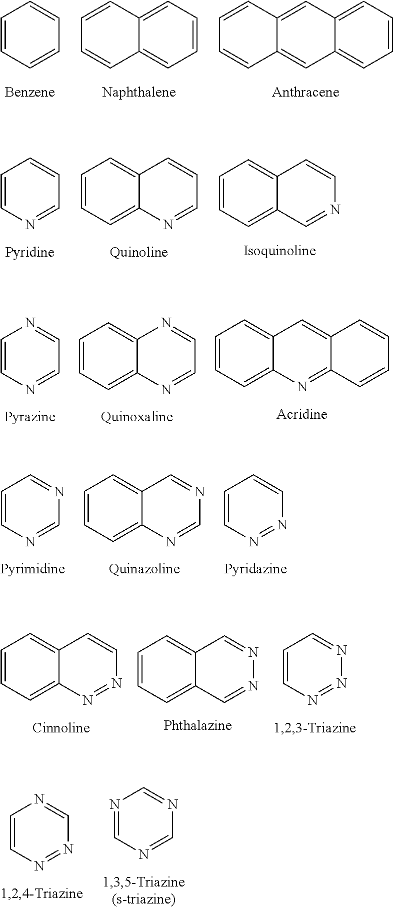

In various embodiments, the direct dye having at least one halogenated aromatic ring has at least one halogenated aryl group. In various embodiments, the direct dye having at least one halogenated aromatic ring has at least one halogenated phenyl group.

### Direct Dyes Generally

Nonlimiting examples of direct dyes generally (i.e., direct dyes comprising at least one halogenated aromatic ring and direct dyes without halogenated aromatic ringa) include azo direct dyes; anthraquinone and anthraquinone derivatives; (poly)methine dyes such as cyanins, hemicyanins and styryls; carbonyl dyes; azine dyes; nitro(hetero)aryl dyes; tri(hetero)arylmethane dyes; porphyrin dyes; phthalocyanin dyes, and natural direct dyes, alone or as mixtures. The direct dyes may be cationic or anionic.

Many direct dyes are cationic direct dyes. Mention may be made of the hydrazono cationic dyes of formulas (Va) and (V′a), the azo cationic dyes (VIa) and (VI′a) and the diazo cationic dyes (VIIa) below:

| | |
|---|---|
| Het⁺—C(R^{a})═N—N(R^{b})—Ar, An⁻ | (Va) |
| Het⁺—N(R^{a})—N═C(R^{b})—Ar, An⁻ | (V′a) |
| Het⁺—N═N—Ar, An⁻ | (VIa) |
| Ar⁺—N═N—Ar″, An⁻ | (VI′a) and |
| Het⁺—N═N—Ar′—N═N—Ar, An⁻ | (VIIa) |

in which formulas (Va), (V′a), (VIa), (VI′a) and (VIIa):
Het⁺ represents a cationic heteroaryl radical, preferably bearing an endocyclic cationic charge, such as imidazolium, indolium or pyridinium, optionally substituted preferentially with one or more (C₁-C₈) alkyl groups such as methyl;
Ar⁺ representing an aryl radical, such as phenyl or naphthyl, bearing an exocyclic cationic charge, preferentially ammonium, particularly tri(C₁-C₈)alkylammonium such as trimethylammonium;
Ar represents an aryl group, especially phenyl, which is optionally substituted, preferentially with one or more electron-donating groups such as i) optionally substituted (C₁-C₈)alkyl, ii) optionally substituted (C₁-C₈)alkoxy, iii) (di)(C₁-C₈)(alkyl)amino optionally substituted on the alkyl group(s) with a hydroxyl group, iv) aryl(C₁-C₈)alkylamino, v) optionally substituted N—(C₁-C₈)alkyl-N-aryl(C₁-C₈)alkylamino or alternatively Ar represents a julolidine group;
Ar′ is an optionally substituted divalent (hetero)arylene group such as phenylene, particularly para-phenylene, or naphthalene, which are optionally substituted, preferentially with one or more groups (C₁-C₈)alkyl, hydroxyl or (C₁-C₈)alkoxy
Ar″ is an optionally substituted (hetero)aryl group such as phenyl or pyrazolyl, which are optionally substituted, preferentially with one or more groups (C₁-C₈)alkyl, hydroxyl, (di)(C₁-C₈)(alkyl)amino, (C₁-C₈)alkoxy or phenyl;
R^{a} and R^{b}, which may be identical or different, represent a hydrogen atom or a group (C₁-C₈)alkyl, which is optionally substituted, preferentially with a hydroxyl group;
or alternatively the substituent R^{a} with a substituent of Het⁺ and/or R_{b} with a substituent of Ar and/or R^{a} with R_{b} form, together with the atoms that bear them, a (hetero)cycloalkyl;
particularly, R^{a} and R_{b} represent a hydrogen atom or a group (C₁-C₄)alkyl, which is optionally substituted with a hydroxyl group;
An⁻ represents an anionic counter-ion such as mesylate or halide. In particular, mention may be made of the azo and hydrazono cationic dyes bearing an endocyclic cationic charge of formulae (Va), (V′a) and (VIa) as defined previously. More particularly those of formulae (Va), (V′a) and (VIa) derived from the dyes described in patent applications WO 95/15144, WO 95/01772 and EP-714954, which are incorporated herein by reference in their entirety.

In some cases, the cationic part is derived from the following derivatives:

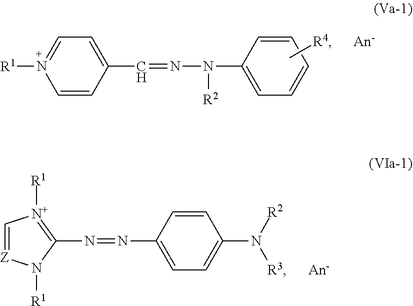

formulae (V-1) and (VI-1) with:
R¹ representing a (C₁-C₄) alkyl group such as methyl;
R² and R³, which are identical or different, represent a hydrogen atom or a (C₁-C₄)alkyl group, such as methyl; and
R⁴ represents a hydrogen atom or an electron-donating group such as optionally substituted (C₁-C₈)alkyl, optionally substituted (C₁-C₈)alkoxy, or (di)(C₁-C₈)(alkyl)amino optionally substituted on the alkyl group(s) with a hydroxyl group; particularly, R⁴ is a hydrogen atom,
Z represents a CH group or a nitrogen atom, preferentially CH;
An⁻ represents an anionic counter-ion such as mesylate or halide.

Particularly, the dye of formulae (Va-1) and (VIa-1) is chosen from Basic Red 51, Basic Yellow 87 and Basic Orange 31 or derivatives thereof:

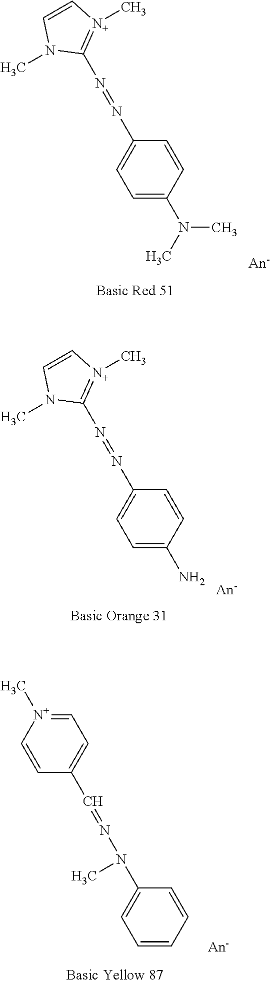

Non-limiting examples of cationic dyes include Basic Blue 6, Basic Blue 7, Basic Blue 9, Basic Blue 26, Basic Blue 41, Basic Blue 99, Basic Brown 4, Basic Brown 16, Basic Brown 17, Natural Brown 7, Basic Green, Basic Orange 31, 1, Basic Red 2, Basic Red 12 Basic Red 22, Basic Red 76 Basic Red 51, Basic Violet 1, Basic Violet 2, Basic Violet 3, Basic Violet 10, Basic Violet 14, Basic Yellow 57 and Basic Yellow 87.

Non-limiting examples anionic dyes include Acid Black 1, Acid Blue 1, Acid Blue 3, Food Blue 5, Acid Blue 7, Acid Blue 9, Acid Blue 74, Acid Orange 3, Acid Orange 6, Acid Orange 7, Acid Orange 10, Acid Red 1, Acid Red 14, Acid Red 18, Acid Red 27, Acid Red 50, Acid Red 52, Acid Red 73, Acid Red 87, Acid Red 88, Acid Red 92, Acid Red 155, Acid Red 180, Acid Violet 9, Acid Violet 43, Acid Violet 49, Acid Yellow 1, Acid Yellow 23, Acid Yellow 3, Food Yellow No. 8, D&C Brown No. 1, D&C Green No. 5, D&C Green No. 8, D&C Orange No. 4, D&C Orange No. 10, D&C Orange No. 11, D&C Red No. 21, D&C Red No. 27, D&C Red No. 33, D&C Violet 2 (Ext Violet 2), D&C Yellow No. 7, D&C Yellow No. 8, D&C Yellow No. 10, FD&C Red 2, FD&C Red 40, FD&C Red No. 4, FD&C Yellow No. 6, FD&C Blue 1, Food Black 1, Food Black 2, Disperse Black 9 and Disperse Violet 1 and their alkali metal salts such as sodium and/or potassium.

Non-limiting examples of nitro dyes include HC Blue No. 2, HC Blue No. 4, HC Blue No. 5, HC Blue No. 6, HC Blue No. 7, HC Blue No. 8, HC Blue No. 9, HC Blue No. 10, HC Blue No. 11, HC Blue No. 12, HC Blue No. 13, HC Blue No. 17, HC Brown No. 1, HC Brown No. 2, HC Green No. 1, HC Orange No. 1, HC Orange No. 2, HC Orange No. 3, HC Orange No. 5, HC Red BN, HC Red No. 1, HC Red No. 3, HC Red No. 7, HC Red No. 8, HC Red No. 9, HC Red No. 10, HC Red No. 11, HC Red No. 13, HC Red No. 54, HC Red No. 14, HC Violet BS, HC Violet No. 1, HC Violet No. 2, HC Yellow No. 2, HC Yellow No. 4, HC Yellow No. 5, HC Yellow No. 6, HC Yellow No. 7, HC Yellow No. 8, HC Yellow No. 9, HC Yellow No. 10, HC Yellow No. 11, HC Yellow No. 12, HC Yellow No. 13, HC Yellow No. 14, HC Yellow No. 15, 2-Amino-6-chloro-4-nitrophenol, picramic acid, 1,2-Diamino-4-nitrobenzol, 1,4-Diamino-2-nitrobenzol, 3-Nitro-4-aminophenol, 1-Hydroxy-2-amino-3-nitrobenzol and 2-hydroxyethylpicramic acid.

### Nonionic Surfactants

Nonionic surfactants are compounds well known in themselves (see, e.g., in this regard, “Handbook of Surfactants” by M. R. Porter, Blackie & Son publishers (Glasgow and London), 1991, pp. 116-178), which is incorporated herein by reference in its entirety.

The nonionic surfactant can be, for example, selected from alcohols, alpha-diols, alkylphenols and esters of fatty acids, these compounds being ethoxylated, propoxylated or glycerolated and having at least one fatty chain comprising, for example, from 8 to 18 carbon atoms, it being possible for the number of ethylene oxide or propylene oxide groups to range from 2 to 50, and for the number of glycerol groups to range from 1 to 30. Maltose derivatives may also be mentioned. Non-limiting mention may also be made of copolymers of ethylene oxide and/or of propylene oxide; condensates of ethylene oxide and/or of propylene oxide with fatty alcohols; polyethoxylated fatty amides comprising, for example, from 2 to 30 mol of ethylene oxide; polyglycerolated fatty amides comprising, for example, from 1.5 to 5 glycerol groups, such as from 1.5 to 4; ethoxylated fatty acid esters of sorbitan comprising from 2 to 30 mol of ethylene oxide; ethoxylated oils from plant origin; fatty acid esters of sucrose; fatty acid esters of polyethylene glycol; polyethoxylated fatty acid mono or diesters of glycerol (C₆-C₂₄)alkylpolyglycosides; N-(C₆-C₂₄)alkylglucamine derivatives, amine oxides such as (C₁₀-C₁₄)alkylamine oxides or N-(C₁₀-C₁₄)acylaminopropylmorpholine oxides; and a mixture thereof.

The nonionic surfactants may preferably be chosen from polyoxyalkylenated or polyglycerolated nonionic surfactants. The oxyalkylene units are more particularly oxyethylene or oxypropylene units, or a combination thereof, and are preferably oxyethylene units.

Examples of oxyalkylenated nonionic surfactants that may be mentioned include: oxyalkylenated (C₈-C₂₄)alkylphenols, saturated or unsaturated, linear or branched, oxyalkylenated C₈-C₃₀ alcohols, saturated or unsaturated, linear or branched, oxyalkylenated C₈-C₃₀ amides, esters of saturated or unsaturated, linear or branched, C₈-C₃₀ acids and of polyethylene glycols, polyoxyalkylenated esters of saturated or unsaturated, linear or branched, C₈-C₃₀ acids and of sorbitol, saturated or unsaturated, oxyalkylenated plant oils, condensates of ethylene oxide and/or of propylene oxide, inter alia, alone or as mixtures.

The surfactants preferably contain a number of moles of ethylene oxide and/or of propylene oxide of between 2 and 100 and most preferably between 2 and 50.

In accordance with one preferred embodiment of the invention, the oxyalkylenated nonionic surfactants are chosen from oxyethylenated C₈-C₃₀ alcohols.

Examples of ethoxylated fatty alcohols (or C₈-C₃₀ alcohols) that may be mentioned include the adducts of ethylene oxide with lauryl alcohol, especially those containing from 9 to 50 oxyethylene groups and more particularly those containing from 10 to 25 oxyethylene groups (Laureth-10 to Laureth-25); the adducts of ethylene oxide with behenyl alcohol, especially those containing from 9 to 50 oxyethylene groups (Beheneth-9 to Beheneth-50); the adducts of ethylene oxide with cetearyl alcohol (mixture of cetyl alcohol and stearyl alcohol), especially those containing from 10 to 30 oxyethylene groups (Ceteareth-10 to Ceteareth-30); the adducts of ethylene oxide with cetyl alcohol, especially those containing from 10 to 30 oxyethylene groups (Ceteth-10 to Ceteth-30); the adducts of ethylene oxide with stearyl alcohol, especially those containing from 10 to 30 oxyethylene groups (Steareth-10 to Steareth-30); the adducts of ethylene oxide with isostearyl alcohol, especially those containing from 10 to 50 oxyethylene groups (Isosteareth-10 to Isosteareth-50); and a mixture thereof.

As examples of polyglycerolated nonionic surfactants, polyglycerolated C₈-C₄₀ alcohols are preferably used.

In particular, the polyglycerolated C₈-C₄₀ alcohols correspond to the following formula:

RO—[CH₂—CH(CH₂OH)—O]ₘ—H or RO—[CH(CH₂OH)—CH₂O]ₘ—H

in which R represents a linear or branched C₈-C₄₀ and preferably C₈-C₃₀ alkyl or alkenyl radical, and m represents a number ranging from 1 to 30 and preferably from 1.5 to 10.

As examples of compounds that are suitable in the context of the invention, mention may be made of lauryl alcohol containing 4 mol of glycerol (INCI name: Polyglyceryl-4 Lauryl Ether), lauryl alcohol containing 1.5 mol of glycerol, oleyl alcohol containing 4 mol of glycerol (INCI name: Polyglyceryl-4 Oleyl Ether), oleyl alcohol containing 2 mol of glycerol (INCI name: Polyglyceryl-2 Oleyl Ether), cetearyl alcohol containing 2 mol of glycerol, cetearyl alcohol containing 6 mol of glycerol, oleocetyl alcohol containing 6 mol of glycerol, and octadecanol containing 6 mol of glycerol.

The alcohol may represent a mixture of alcohols in the same way that the value of m represents a statistical value, which means that, in a commercial product, several species of polyglycerolated fatty alcohol may coexist in the form of a mixture.

According to one of the embodiments according to the present invention, the nonionic surfactant may be selected from esters of polyols with fatty acids with a saturated or unsaturated chain containing for example from 8 to 24 carbon atoms, preferably 12 to 22 carbon atoms, and alkoxylated derivatives thereof, preferably with a number of alkyleneoxide of from 10 to 200, and more preferably from 10 to 100, such as glyceryl esters of a C₈-C₂₄, preferably C₁₂-C₂₂, fatty acid or acids and alkoxylated derivatives thereof, preferably with a number of alkyleneoxide of from 10 to 200, and more preferably from 10 to 100; polyethylene glycol esters of a C₈-C₂₄, preferably C₁₂-C₂₂, fatty acid or acids and alkoxylated derivatives thereof, preferably with a number of alkyleneoxide of from 10 to 200, and more preferably from 10 to 100; sorbitol esters of a C₈-C₂₄, preferably C₁₂-C₂₂, fatty acid or acids and alkoxylated derivatives thereof, preferably with a number of alkyleneoxide of from 10 to 200, and more preferably from 10 to 100; sugar (sucrose, glucose, alkylglycose) esters of a C₈-C₂₄, preferably C₁₂-C₂₂, fatty acid or acids and alkoxylated derivatives thereof, preferably with a number of alkyleneoxide of from 10 to 200, and more preferably from 10 to 100; ethers of fatty alcohols; ethers of sugar and a C₈-C₂₄, preferably C₁₂-C₂₂, fatty alcohol or alcohols; and a mixture thereof.

Examples of ethoxylated fatty esters that may be mentioned include the adducts of ethylene oxide with esters of lauric acid, palmitic acid, stearic acid or behenic acid, and a mixture thereof, especially those containing from 9 to 100 oxyethylene groups, such as PEG-9 to PEG-50 laurate; PEG-9 to PEG-50 palmitate; PEG-9 to PEG-50 stearate; PEG-9 to PEG-50 palmitostearate; PEG-9 to PEG-50 behenate; polyethylene glycol 100 EO monostearate; and a mixture thereof.

As glyceryl esters of fatty acids, glyceryl stearate (glyceryl mono-, di- and/or tristearate) (glyceryl stearate) or glyceryl ricinoleate and a mixture thereof can in particular be cited.

As glyceryl esters of C₈-C₂₄ alkoxylated fatty acids, polyethoxylated glyceryl stearate (glyceryl mono-, di- and/or tristearate) such as PEG-20 glyceryl stearate can for example be cited.

Mixtures of these surfactants, such as for example the product containing glyceryl stearate and PEG-100 stearate, marketed under the name ARLACEL 165 by Croda, and a product containing glyceryl stearate (glyceryl mono- and distearate) and potassium stearate, can also be used.

The sorbitol esters of C₈-C₂₄ fatty acids and alkoxylated derivatives thereof can be selected from sorbitan palmitate, sorbitan trioleate and esters of fatty acids and alkoxylated sorbitan containing for example from 20 to 100 EO, such as for example polyethylene sorbitan trioleate (polysorbate 85) or the compounds marketed under the trade names Tween 20 or Tween 60 by Croda.

As esters of fatty acids and glucose or alkylglucose, in particular glucose palmitate, alkylglucose sesquistearates such as methylglucose sesquistearate, alkylglucose palmitates such as methylglucose or ethylglucose palmitate, methylglucoside fatty esters and more specifically the diester of methylglucoside and oleic acid (Methyl glucose dioleate), the mixed ester of methylglucoside and the mixture oleic acid/hydroxystearic acid (Methyl glucose dioleate/hydroxystearate), the ester of methylglucoside and isostearic acid (Methyl glucose isostearate), the ester of methylglucoside and lauric acid (Methyl glucose laurate), the mixture of monoester and diester of methylglucoside and isostearic acid (Methyl glucose sesqui-isostearate), the mixture of monoester and diester of methylglucoside and stearic acid (Methyl glucose sesquistearate) and in particular the product marketed under the name Glucate SS by Lubrizol, and a mixture thereof can be cited.

As ethoxylated ethers of fatty acids and glucose or alkylglucose, ethoxylated ethers of fatty acids and methylglucose, and in particular the polyethylene glycol ether of the diester of methylglucose and stearic acid with about 20 moles of ethylene oxide (PEG-20 methyl glucose distearate) such as the product marketed under the name GLUCAM E-20 DISTEARATE by Lubrizol, the polyethylene glycol ether of the mixture of monoester and diester of methyl-glucose and stearic acid with about 20 moles of ethylene oxide (PEG-20 methyl glucose sesquistearate) and in particular the product marketed under the name GLUCAMATE SSE-20 by Lubrizol, and a mixture thereof, can for example be cited.

As sucrose esters, saccharose palmito-stearate, saccharose stearate and saccharose monolaurate can for example be cited.

As sugar ethers, alkylpolyglucosides can be used, and for example decylglucoside such as the product marketed under the name MYDOL 10 by Kao Chemicals, the product marketed under the name PLATAREN 2000 by BASF, and the product marketed under the name ORAMIX NS 10 by Seppic, caprylyl/capryl glucoside such as the product marketed under the name ORAMIX CG 110 by Seppic or under the name LUTENSOL GD 70 by BASF, laurylglucoside such as the products marketed under the names PLANTAREN 1200 N and PLANTACARE 1200 by BASF, coco-glucoside such as the product marketed under the name PLANTACARE 818/UP by BASF, cetostearyl glucoside possibly mixed with cetostearyl alcohol, marketed for example under the name MONTANOV 68 by Seppic, under the name TEGO-CARE CG90 by Evonik, arachidyl glucoside, for example in the form of the mixture of arachidyl and behenyl alcohols and arachidyl glucoside marketed under the name MONTANOV 202 by Seppic, cocoylethylglucoside, for example in the form of the mixture (35/65) with cetyl and stearyl alcohols, marketed under the name MONTANOV 82 by Seppic, and a mixture thereof can in particular be cited.

Mixtures of glycerides of alkoxylated plant oils such as mixtures of ethoxylated (200 EO) palm and copra (7 EO) glycerides can also be cited.

The nonionic surfactant may be selected from the group consisting of PEG-7 glyceryl cocoate, PEG-20 methylglucoside sesquistearate, PEG-20 glyceryl tri-isostearate, PG-5 dioleate, PG-4 diisostearate, PG-10 isostearate, PEG-8 isostearate, and PEG-60 hydrogenated castor oil.

Mixtures of these oxyethylenated derivatives of fatty alcohols and of fatty esters may also be used.

In some case, the nonionic surfactant is a fatty alkanolamide. Non-limiting examples of fatty alkanolamides that may be used include cocamide MEA, cocamide DEA, soyamide DEA, lauramide DEA, oleamide MIPA, stearamide MEA, myristamide DEA, stearamide DEA, oleylamide DEA, tallowamide DEA lauramide MIPA, tallowamide MEA, isostearamide DEA, isostearamide MEA, and a mixture thereof.

In some cases, the nonionic surfactant is an oxyethylenated amide such as PEG-4 rapeseedamide.

### Cationic Conditioning Polymers

Non-limiting examples of cationic polymers include copolymers of 1-vinyl-2-pyrrolidine and 1-vinyl-3-methyl-imidazolium salt (e.g., chloride salt) (referred to as Polyquaternium-16); copolymers of 1-vinyl-2-pyrrolidine and dimethylaminoethyl methacrylate (referred to as Polyquaternium-11); cationic diallyl quaternary ammonium-containing polymer including, for example, dimethyldiallyammonium chloride homopolymer and copolymers of acrylamide and dimethyldiallyammonium chloride (referred to as Polyquaternium-6 and Polyquaternium-7); polysaccharide polymers, such as cationic cellulose derivatives and cationic starch derivatives. Cationic cellulose is available as salts of hydroxyethyl cellulose reacted with trimethyl ammonium substituted epoxide (referred to as Polyquaternium-10). Another type of cationic cellulose includes the polymeric quaternary ammonium salts of hydroxyethyl cellulose reacted with lauryl dimethyl ammonium-substituted epoxide (referred to as Polyquaternium-24). Additionally or alternatively, the cationic conditioning polymers may include or be chosen from cationic guar gum derivatives, such as guar hydroxypropyltrimonium chloride.

In certain embodiments, the one or more cationic conditioning polymers include cationic polysaccharide polymers, such as cationic cellulose, cationic starch, and cationic guar gum. In the context of the instant disclosure cationic polysaccharide polymers include cationic polysaccharides and polysaccharide derivatives (e.g., derivatized to be cationic), for example, resulting in cationic cellulose (cellulose derivatized to be cationic), cationic starch (derivatized to be cationic), cationic guar (guar derivatized to be cationic).

Non-limiting examples of cationic celluloses include hydroxyethylcellulose (also known as HEC), hydroxymethylcellulose, methylhydroxyethylcellulose, hydroxypropylcellulose (also known as HPC), hydroxybutylcellulose, hydroxyethylmethylcellulose (also known as methyl hydroxyethylcellulose) and hydroxypropylmethylcellulose (also known as HPMC), cetyl hydroxyethylcellulose, polyquaternium-10, polyquaternium-24, and mixtures thereof, preferably polyquaternium-10, polyquaternium-24, and mixtures thereof.

Non-limiting examples of cationic guar include guar hydroxypropyltrimonium chloride, hydroxypropyl guar hydroxypropyltrimonium chloride,

Non-limiting examples of cationic starch include starch hydroxypropyltrimonium chloride, hydroxypropyl oxidized starch PG trimonium chloride, and a mixture thereof.

In various embodiments, the one or more cationic conditioning polymers are chosen from polyquaterniums. Nonlimiting examples include Polyquaternium-1 (ethanol, 2,2′,2″-nitrilotris-, polymer with 1,4-dichloro-2-butene and N,N,N′,N′-tetramethyl-2-butene-1,4-diamine), Polyquaternium-2, (poly[bis(2-chloroethyl) ether-alt-1,3-bis[3-(dimethylamino)propyl]urea]), Polyquaternium-4, (hydroxyethyl cellulose dimethyl diallylammonium chloride copolymer; Diallyldimethylammonium chloride-hydroxyethyl cellulose copolymer), Polyquaternium-5 (copolymer of acrylamide and quaternized dimethylammoniumethyl methacrylate), Polyquaternium-6 (poly(diallyldimethylammonium chloride)), Polyquaternium-7 (copolymer of acrylamide and diallyldimethylammonium chloride), Polyquaternium-8 (copolymer of methyl and stearyl dimethylaminoethyl ester of methacrylic acid, quaternized with dimethylsulphate), Polyquaternium-9 (homopolymer of N,N-(dimethylamino)ethyl ester of methacrylic acid, quaternized with bromomethane), Polyquaternium-10 (quaternized hydroxyethyl cellulose), Polyquaternium-11 (copolymer of vinylpyrrolidone and quaternized dimethylaminoethyl methacrylate), Polyquaternium-12 (ethyl methacrylate/abietyl methacrylate/diethylaminoethyl methacrylate copolymer quaternized with dimethyl sulfate), Polyquaternium-13 (ethyl methacrylate/oleyl methacrylate/diethylaminoethyl methacrylate copolymer quaternized with dimethyl sulfate), Polyquaternium-14 (trimethylaminoethylmethacrylate homopolymer), Polyquaternium-15 (acrylamide-dimethylaminoethyl methacrylate methyl chloride copolymer), Polyquaternium-16 (copolymer of vinylpyrrolidone and quaternized vinylimidazole), Polyquaternium-17 (adipic acid, dimethylaminopropylamine and dichloroethylether copolymer), Polyquaternium-18 (azelanic acid, dimethylaminopropylamine and dichloroethylether copolymer), Polyquaternium-19 (copolymer of polyvinyl alcohol and 2,3-epoxypropylamine), Polyquaternium-20 (copolymer of polyvinyl octadecyl ether and 2,3-epoxypropylamine), Polyquaternium-22 (copolymer of acrylic acid and diallyldimethylammonium chloride), Polyquaternium-24 (quaternary ammonium salt of hydroxyethyl cellulose reacted with a lauryl dimethyl ammonium substituted epoxide), Polyquaternium-27 (block copolymer of Polyquaternium-2 and Polyquaternium-17), Polyquaternium-28 (copolymer of vinylpyrrolidone and methacrylamidopropyl trimethylammonium), Polyquaternium-29 (chitosan modified with propylen oxide and quaternized with epichlorhydrin), Polyquaternium-30 (ethanaminium, N-(carboxymethyl)-N,N-dimethyl-2-[(2-methyl-1-oxo-2-propen-1-yl)oxy]-, inner salt, polymer with methyl 2-methyl-2-propenoate), Polyquaternium-31 (N,N-dimethylaminopropyl-N-acrylamidine quaternized with diethylsulfate bound to a block of polyacrylonitrile), Polyquaternium-32 (poly(acrylamide 2-methacryloxyethyltrimethyl ammonium chloride)), Polyquaternium-33 (copolymer of trimethylaminoethylacrylate salt and acrylamide), Polyquaternium-34 (copolymer of 1,3-dibromopropane and N,N-diethyl-N′,N′-dimethyl-1,3-propanediamine), Polyquaternium-35 (methosulphate of the copolymer of methacryloyloxyethyltrimethylammonium and of methacryloyloxyethyldimethylacetylammonium), Polyquaternium-36 (copolymer of N,N-dimethylaminoethylmethacrylate and buthylmethacrylate, quaternized with dimethylsulphate), Polyquaternium-37 (poly(2-methacryloxyethyltrimethylammonium chloride)), Polyquaternium-39 (terpolymer of acrylic acid, acrylamide and diallyldimethylammonium Chloride), Polyquaternium-42 (poly[oxyethylene(dimethylimino)ethylene (dimethylimino)ethylene dichloride]), Polyquaternium-43 (copolymer of acrylamide, acrylamidopropyltrimonium chloride, 2-amidopropylacrylamide sulfonate and dimethylaminopropylamine), Polyquaternium-44 (3-Methyl-1-vinylimidazolium methyl sulfate-N-vinylpyrrolidone copolymer), Polyquaternium-45 (copolymer of (N-methyl-N-ethoxyglycine)methacrylate and N,N-dimethylaminoethylmethacrylate, quaternized with dimethyl sulphate), Polyquaternium-46 (terpolymer of vinylcaprolactam, vinylpyrrolidone, and quaternized vinylimidazole), Polyquaternium-47 (terpolymer of acrylic acid, methacrylamidopropyl trimethylammonium chloride, and methyl acrylate), and/or Polyquaternium-67.

In various embodiments, the one or more cationic conditioning polymers are chosen from cationic cellulose derivatives, quaternized hydroxyethyl cellulose (e.g., polyquaternium-10), cationic starch derivatives, cationic guar gum derivatives, copolymers of acrylamide and dimethyldiallyammonium chloride (e.g., polyquaternium-7), polyquaterniums, and a mixture thereof. For example, the cationic polymer(s) may be selected from polyquaterniums, for example, polyquaterniums selected from polyquaternium-4, polyquaternium-5, polyquaternium-6, polyquaternium-7, polyquaternium-10, polyquaternium-22, polyquaternium-37, polyquaternium-39, polyquaternium-47, polyquaternium-53, polyquaternium-67 and a mixture thereof. A combination of two or more polyquaterniums can be useful. A particularly preferred and useful cationic polymer is polyquaternium-10.

The cationic polymers may be a polyquaternium. In certain embodiments, the cationic surfactants may be polyquaterniums selected from polyquaternium-1, polyquaternium-2, polyquaternium-3, polyquaternium-4, polyquaternium-5, polyquaternium-6, polyquaternium-7, polyquaternium-8, polyquaternium-9, polyquaternium-10, polyquaternium-11, polyquaternium-12, polyquaternium-13, polyquaternium-14, polyquaternium-15, polyquaternium-16, polyquaternium-17, polyquaternium-18, polyquaternium-19, polyquaternium-20, polyquaternium-21, polyquaternium-22, polyquaternium-23, polyquaternium-24, polyquaternium-25, polyquaternium-26, polyquaternium-27, polyquaternium-28, polyquaternium-29, polyquaternium-30, polyquaternium-40, polyquaternium-41, polyquaternium-42, polyquaternium-43, polyquaternium-44, polyquaternium-45, polyquaternium-46, polyquaternium-47, polyquaternium-48, polyquaternium-49, polyquaternium-50, polyquaternium-51, polyquaternium-52, polyquaternium-53, polyquaternium-54, polyquaternium-55, polyquaternium-56, polyquaternium-57, polyquaternium-58, polyquaternium-59, polyquaternium-60, polyquaternium-61, polyquaternium-62, polyquaternium-63, polyquaternium-64, polyquaternium-65, polyquaternium-66, polyquaternium-67, etc. In some cases, preferred polyquaternium compounds include polyquaternium-10, polyquaternium-11, polyquaternium-67, and a mixture thereof.

In some embodiments, the one or more cationic conditioning polymers are chosen from cationic proteins and cationic protein hydrolysates (e.g., hydroxypropyltrimonium hydrolyzed wheat protein), quaternary diammonium polymers (e.g., hexadimethrine chloride), copolymers of acrylamide and dimethyldiallyammonium chloride, and mixtures thereof.

The cationic conditioning polymers may be homopolymers or formed from two or more types of monomers. The molecular weight of the polymer may be between 5,000 and 10,000,000, typically at least 10,000, and preferably in the range 100,000 to about 2,000,000. These polymers will typically have cationic nitrogen containing groups such as quaternary ammonium or protonated amino groups, or a mixture thereof.

The cationic charge density is suitably at least 0.1 meq/g, preferably above 0.8 or higher. In some instances, the cationic charge density does not exceed 3 meq/g, or does not exceed 2 meq/g. The charge density can be measured using the Kjeldahl method and can be within the above limits at the desired pH of use, which will in general be from about 3 to 9 and preferably between 4 and 8.

The cationic nitrogen-containing group will generally be present as a substituent on a fraction of the total monomer units of the cationic conditioning polymer. Thus when the polymer is not a homopolymer it can contain spacer non-cationic monomer units.

Suitable cationic conditioning polymers include, for example, copolymers of vinyl monomers having cationic amine or quaternary ammonium functionalities with water soluble spacer monomers such as (meth)acrylamide, alkyl and dialkyl (meth)acrylamides, alkyl (meth)acrylate, vinyl caprolactone and vinyl pyrrolidine. The alkyl and dialkyl substituted monomers preferably have C₁-C₇ alkyl groups, more preferably C₁-C₃ alkyl groups. Other suitable spacers include vinyl esters, vinyl alcohol, maleic anhydride, propylene glycol and ethylene glycol.

The cationic amines can be primary, secondary or tertiary amines, depending upon the particular species and the pH of the composition.

Amine substituted vinyl monomers and amines can be polymerized in the amine form and then converted to ammonium by quaternization.

Suitable cationic amino and quaternary ammonium monomers include, for example, vinyl compounds substituted with dialkyl aminoalkyl acrylate, dialkylamino alkylmethacrylate, monoalkylaminoalkyl acrylate, monoalkylaminoalkyl methacrylate, trialkyl methacryloxyalkyl ammonium salt, trialkyl acryloxyalkyl ammonium salt, diallyl quaternary ammonium salts, and vinyl quaternary ammonium monomers having cyclic cationic nitrogen-containing rings such as pyridinium, imidazolium, and quaternized pyrrolidine, e.g., alkyl vinyl imidazolium, and quaternized pyrrolidine, e.g., alkyl vinyl imidazolium, alkyl vinyl pyridinium, alkyl vinyl pyrrolidine salts. The alkyl portions of these monomers are preferably lower alkyls such as the C₁-C₃ alkyls, more preferably C₁ and C₂ alkyls.

Suitable amine-substituted vinyl monomers include dialkylaminoalkyl acrylate, dialkylaminoalkyl methacrylate, dialkylaminoalkyl acrylamide, and dialkylaminoalkyl methacrylamide, wherein the alkyl groups are preferably C₁-C₇ hydrocarbyls, more preferably C₁-C₃, alkyls.

The cationic conditioning polymers can comprise mixtures of monomer units derived from amine- and/or quaternary ammonium-substituted monomer and/or compatible spacer monomers.

Suitable cationic conditioning polymers include, for example: copolymers of 1-vinyl-2-pyrrolidine and 1-vinyl-3-methyl-imidazolium salt (e.g., Chloride salt) (referred to as Polyquaternium-16) such as those commercially available from BASF under the LUVIQUAT tradename (e.g., LUVIQUAT FC 370); copolymers of 1-vinyl-2-pyrrolidine and dimethylaminoethyl methacrylate (referred to as Polyquaternium-11) such as those commercially from Gar Corporation (Wayne, N.J., USA) under the GAFQUAT tradename (e.g., GAFQUAT 755N); and cationic diallyl quaternary ammonium-containing polymer including, for example, dimethyldiallyammonium chloride homopolymer and copolymers of acrylamide and dimethyldiallyammonium chloride (referred to as Polyquaternium-6 and Polyquaternium-7).

Other cationic conditioning polymers that can be used include polysaccharide polymers, such as cationic cellulose derivatives and cationic starch derivatives. Cationic cellulose is available from Amerchol Corp. (Edison, N.J., USA) in their Polymer JR (trade mark) and LR (trade mark) series of polymers, as salts of hydroxyethyl cellulose reacted with trimethyl ammonium substituted epoxide (referred to as Polyquaternium-10). Another type of cationic cellulose includes the polymeric quaternary ammonium salts of hydroxyethyl cellulose reacted with lauryl dimethyl ammonium-substituted epoxide (referred to as Polyquaternium-24). These materials are available from Amerchol Corp. (Edison, N.J., USA) under the tradename Polymer LM-200.

Other cationic conditioning polymers that can be used include cationic guar gum derivatives, such as guar hydroxypropyltrimonium chloride.

Polyquaterniums include Polyquaternium-1 (ethanol, 2,2′,2″-nitrilotris-, polymer with 1,4-dichloro-2-butene and N,N,N′,N′-tetramethyl-2-butene-1,4-diamine), Polyquaternium-2, (poly[bis(2-chloroethyl) ether-alt-1,3-bis[3-(dimethylamino)propyl]urea]), Polyquaternium-4, (hydroxyethyl cellulose dimethyl diallylammonium chloride copolymer; Diallyldimethylammonium chloride-hydroxyethyl cellulose copolymer), Polyquaternium-5 (copolymer of acrylamide and quaternized dimethylammoniumethyl methacrylate), polyquaternium-6 (poly(diallyldimethylammonium chloride)), Polyquaternium-7 (copolymer of acrylamide and diallyldimethylammonium chloride), Polyquaternium-8 (copolymer of methyl and stearyl dimethylaminoethyl ester of methacrylic acid, quaternized with dimethylsulphate), Polyquaternium-9 (homopolymer of N,N-(dimethylamino)ethyl ester of methacrylic acid, quaternized with bromomethane), Polyquaternium-10 (quaternized hydroxyethyl cellulose), polyquaternium-11 (copolymer of vinylpyrrolidone and quaternized dimethylaminoethyl methacrylate), Polyquaternium-12 (ethyl methacrylate/abietyl methacrylate/diethylaminoethyl methacrylate copolymer quaternized with dimethyl sulfate), Polyquaternium-13 (ethyl methacrylate/oleyl methacrylate/diethylaminoethyl methacrylate copolymer quaternized with dimethyl sulfate), Polyquaternium-14 (trimethylaminoethylmethacrylate homopolymer), Polyquaternium-15 (acrylamide-dimethylaminoethyl methacrylate methyl chloride copolymer), Polyquaternium-16 (copolymer of vinylpyrrolidone and quaternized vinylimidazole), Polyquaternium-17 (adipic acid, dimethylaminopropylamine and dichloroethylether copolymer), Polyquaternium-18 (azelanic acid, dimethylaminopropylamine and dichloroethylether copolymer), Polyquaternium-19 (copolymer of polyvinyl alcohol and 2,3-epoxypropylamine), Polyquaternium-20 (copolymer of polyvinyl octadecyl ether and 2,3-epoxypropylamine), Polyquaternium-22 (copolymer of acrylic acid and diallyldimethylammonium chloride), Polyquaternium-24 (quaternary ammonium salt of hydroxyethyl cellulose reacted with a lauryl dimethyl ammonium substituted epoxide), Polyquaternium-27 (block copolymer of Polyquaternium-2 and Polyquaternium-17), Polyquaternium-28 (copolymer of vinylpyrrolidone and methacrylamidopropyl trimethylammonium), Polyquaternium-29 (chitosan modified with propylen oxide and quaternized with epichlorhydrin), Polyquaternium-30 (ethanaminium, N-(carboxymethyl)-N,N-dimethyl-2-[(2-methyl-1-oxo-2-propen-1-yl)oxy]-, inner salt, polymer with methyl 2-methyl-2-propenoate), Polyquaternium-31 (N,N-dimethylaminopropyl-N-acrylamidine quaternized with diethylsulfate bound to a block of polyacrylonitrile), Polyquaternium-32 (poly(acrylamide 2-methacryloxyethyltrimethyl ammonium chloride)), Polyquaternium-33 (copolymer of trimethylaminoethylacrylate salt and acrylamide), Polyquaternium-34 (copolymer of 1,3-dibromopropane and N,N-diethyl-N′,N′-dimethyl-1,3-propanediamine), Polyquaternium-35 (methosulphate of the copolymer of methacryloyloxyethyltrimethylammonium and of methacryloyloxyethyldimethylacetylammonium), Polyquaternium-36 (copolymer of N,N-dimethylaminoethylmethacrylate and buthylmethacrylate, quaternized with dimethylsulphate), Polyquaternium-37 (poly(2-methacryloxyethyltrimethylammonium chloride)), Polyquaternium-39 (terpolymer of acrylic acid, acrylamide and diallyldimethylammonium Chloride), Polyquaternium-42 (poly[oxyethylene(dimethylimino)ethylene (dimethylimino)ethylene dichloride]), Polyquaternium-43 (copolymer of acrylamide, acrylamidopropyltrimonium chloride, 2-amidopropylacrylamide sulfonate and dimethylaminopropylamine), Polyquaternium-44 (3-Methyl-1-vinylimidazolium methyl sulfate-N-vinylpyrrolidone copolymer), Polyquaternium-45 (copolymer of (N-methyl-N-ethoxyglycine)methacrylate and N,N-dimethylaminoethylmethacrylate, quaternized with dimethyl sulphate), Polyquaternium-46 (terpolymer of vinylcaprolactam, vinylpyrrolidone, and quaternized vinylimidazole), and Polyquaternium-47 (terpolymer of acrylic acid, methacrylamidopropyl trimethylammonium chloride, and methyl acrylate).

### Thickening Agents

Thickening agents that may be mentioned include the following:

a. Carboxylic acid or carboxylate based homopolymer or co-polymer, which can be linear or crosslinked: These polymers contain one or more monomers derived from acrylic acid, substituted acrylic acids, and salts and esters of these acrylic acids (acrylates) and the substituted acrylic acids. Commercially available polymers include those sold under the trade names CARBOPOL, ACRYSOL, POLYGEL, SOKALAN, CARBOPOL ULTREZ, and POLYGEL. Examples of commercially available carboxylic acid polymers include the carbomers, which are homopolymers of acrylic acid crosslinked with allyl ethers of sucrose or pentaerytritol. The carbomers are available as the CARBOPOL 900 series from B.F. Goodrich (e.g., CARBOPOL 954). In addition, other suitable carboxylic acid polymeric agents include ULTREZ 10 (B.F. Goodrich) and copolymers of C10-30 alkyl acrylates with one or more monomers of acrylic acid, methacrylic acid, or one of their short chain (i.e., C1-4 alcohol) esters, wherein the crosslinking agent is an allyl ether of sucrose or pentaerytritol. These copolymers are known as acrylates/C10-C30 alkyl acrylate crosspolymers and are commercially available as CARBOPOL 1342, CARBOPOL 1382, PEMULEN TR-1, and PEMULEN TR-2, from B.F. Goodrich.

Other suitable carboxylic acid or carboxylate polymeric agents include copolymers of acrylic acid and alkyl C5-C10 acrylate, copolymers of acrylic acid and maleic anhydride, and polyacrylate crosspolymer-6. Polyacrylate Crosspolymer-6 is aviable in the raw material known as SEPIMAX ZEN from Seppic.

Another suitable carboxylic acid or carboxylate polymeric agent includes acrylamidopropyltrimonium chloride/acrylates copolymer, a cationic acrylates copolymer (or a quaternary ammonium compound), available as a raw material known under the tradename of SIMULQUAT HC 305 from Seppic.

In certain embodiments, the carboxylic acid or carboxylate polymer thickeners useful herein are those selected from carbomers, acrylates/C10-C30 alkyl acrylate crosspolymers, polyacrylate crosspolymer-6, acrylamidopropyltrimonium chloride/acrylates copolymer, and mixtures thereof.

b. Celluloses: Non-limiting examples of celluloses include cellulose, carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, and mixtures thereof. In some instances, the cellulose is selected from water soluble cellulose derivatives (for example, carboxymethyl cellulose, methyl cellulose, methylhydroxypropyl cellulose, hydroxyethyl cellulose, hydroxypropyl cellulose, cellulose sulfate sodium salt). Furthermore, in some instance, the cellulose is preferably hydroxypropylcellulose (HPC).

c. Polyvinylpyrrolidone (PVP) and co-polymers: Non-limiting examples include Polyvinylpyrrolidone (PVP), Polyvinylpyrrolidone (PVP)/vinyl acetate copolymer (PVP/VA copolymer), polyvinylpyrrolidone (PVP)/eicosene copolymer, PVP/hexadecene copolymer, etc. Commercially available polyvinylpyrrolidone includes LUVISKOL K30, K85, K90 available from BASF. Commercially available copolymers of vinylpyrrolidone and vinylacetate include LUVISKOL VA37, VA64 available from BASF; copolymers of vinylpyrrolidone, methacrylamide, and vinylimidazole (INCI: VP/Methacrylamide/Vinyl Imidazole Copolymer) is commercially available as LUVISET from BASF. In some instances, PVP and PVP/VA copolymer are preferred.

d. Sucrose esters: Non-limiting examples include sucrose palmitate, sucrose cocoate, sucrose monooctanoate, sucrose monodecanoate, sucrose mono- or dilaurate, sucrose monomyristate, sucrose mono- or dipalmitate, sucrose mono- and distearate, sucrose mono-, di- or trioleate, sucrose mono- or dilinoleate, sucrose pentaoleate, sucrose hexaoleate, sucrose heptaoleate or sucrose octooleate, and mixed esters, such as sucrose palmitate/stearate, and mixtures thereof.

e. Polyglyceryl esters: Non-limiting polyglycerol esters of fatty acids (polygylceryl esters) include those of the following formula:

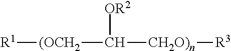

wherein n is from 2 to 20 or from 2 to 10 or from 2 to 5, or is 2, 3, 4, 5, 6, 7, 8, 9, or 10, and R¹, R² and R³ each may independently be a fatty acid moiety or hydrogen, provided that at least one of R¹, R², and R³ is a fatty acid moiety. For instance, R¹, R² and R³ may be saturated or unsaturated, straight or branched, and have a length of C₁-C₄₀, C₁-C₃₀, C₁-C₂₅, or C₁-C₂₀, C₁-C_{16, or} C₁-C₁₀. Additionally, non-limiting examples of nonionic polyglycerol esters of fatty acids include polyglyceryl-4 caprylate/caprate, polyglyceryl-10 caprylate/caprate, polyglyceryl-4 caprate, polyglyceryl-10 caprate, polyglyceryl-4 laurate, polyglyceryl-5 laurate, polyglyceryl-6 laurate, polyglyceryl-10 laurate, polyglyceryl-10 cocoate, polyglyceryl-10 myristate, polyglyceryl-10 oleate, polyglyceryl-10 stearate, and mixtures thereof.

f. C8-24 hydroxyl substituted aliphatic acid and C8-24 conjugated aliphatic acid: Non-limiting examples include conjugated linoleic acid, cis-parinaric acid, trans-7-octadecenoic acid, cis-5,8,11,14,17-eicosapentanoic acid, cis-4,7,10,13,16,19-docosahexenoic acid, columbinic acid, linolenelaidic acid, ricinolaidic acid, stearidonic acid, 2-hydroxystearic acid, alpha-linolenic acid, arachidonic acid, cis-11,14-eicosadienoic acid, linolelaidic acid, monopetroselinic acid, petroselinic acid, ricinoleic acid, trans-vaccenic acid, cis-11,14,17-eicosatrienoic acid, cis-5-eicosenoic acid, cis-8,11,14-eicosatrienoic acid, hexadecatrienoic acid, palmitoleic acid, petroselaidic acid, trans trans farnesol, cis-13,16-docosadienoic acid, cis-vaccenic acid, cis-11-eicosenoic acid, cis-13,16,19-docosatrienoic acid, cis-13-octadecenoic acid, cis-15-octadecanoic acid, cis-7,10,13,16 docosatetraenoic acid, elaidic acid, gamma-linolenic acid, geranic acid, geranyl geranoic acid, linoleic acid, oleic acid, pinolenic acid, trans-13-octadecenoic acid. More preferably, the aliphatic acid comprises 12-hydroxystearic acid, conjugated linoleic acid, or a mixture thereof.

g. Gums: Non-limiting examples of gums include gum arabic, tragacanth gum, karaya gum, guar gum, gellan gum, tara gum, locust bean gum, tamarind gum, xanthan gum, locust bean gum, Seneca gum, sclerotium gum, gellan gum, etc.

### Fatty Compounds Other Than Fatty Alcohols

Fatty acids useful herein include those having from about 10 to about 30 carbon atoms, from about 12 to about 22 carbon atoms, and from about 16 to about 22 carbon atoms. These fatty acids can be straight or branched chain acids and can be saturated or unsaturated. Also included are diacids, triacids, and other multiple acids which meet the carbon number requirement herein. Also included herein are salts of these fatty acids. Nonlimiting examples of fatty acids include lauric acid, palmitic acid, stearic acid, behenic acid, arichidonic acid, oleic acid, isostearic acid, sebacic acid, and a mixture thereof. In some cases, the fatty acids are selected from the group consisting of palmitic acid, stearic acid, and a mixture thereof.

Fatty alcohol derivatives include alkyl ethers of fatty alcohols, alkoxylated fatty alcohols, alkyl ethers of alkoxylated fatty alcohols, esters of fatty alcohols and a mixture thereof. Nonlimiting examples of fatty alcohol derivatives include materials such as methyl stearyl ether; 2-ethylhexyl dodecyl ether; stearyl acetate; cetyl propionate; the ceteth series of compounds such as ceteth-1 through ceteth-45, which are ethylene glycol ethers of cetyl alcohol, wherein the numeric designation indicates the number of ethylene glycol moieties present; the steareth series of compounds such as steareth-1 through 10, which are ethylene glycol ethers of steareth alcohol, wherein the numeric designation indicates the number of ethylene glycol moieties present; ceteareth 1 through ceteareth-10, which are the ethylene glycol ethers of ceteareth alcohol, i.e. a mixture of fatty alcohols containing predominantly cetyl and stearyl alcohol, wherein the numeric designation indicates the number of ethylene glycol moieties present; C1-C30 alkyl ethers of the ceteth, steareth, and ceteareth compounds just described; polyoxyethylene ethers of branched alcohols such as octyldodecyl alcohol, dodecylpentadecyl alcohol, hexyldecyl alcohol, and isostearyl alcohol; polyoxyethylene ethers of behenyl alcohol; PPG ethers such as PPG-9-steareth-3, PPG-11 stearyl ether, PPG8-ceteth-1, and PPG-10 cetyl ether; and a mixture thereof.

Non-limiting examples of polyglycerol esters of fatty acids include those of the following formula:

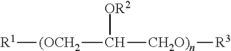

wherein the average value of n is about 3 and R¹, R² and R³ each may independently be a fatty acid moiety or hydrogen, provided that at least one of R¹, R², and R³ is a fatty acid moiety. For instance, R¹, R² and R³ may be saturated or unsaturated, straight or branched, and have a length of C₁-C₄₀, C₁-C₃₀, C₁-C₂₅, or C₁-C₂₀, C₁-C_{16, or} C₁-C₁₀. For example, nonionic polyglycerol esters of fatty acids include polyglyceryl-5 laurate,

The fatty acid derivatives are defined herein to include fatty acid esters of the fatty alcohols as defined above, fatty acid esters of the fatty alcohol derivatives as defined above when such fatty alcohol derivatives have an esterifiable hydroxyl group, fatty acid esters of alcohols other than the fatty alcohols and the fatty alcohol derivatives described above, hydroxy-substituted fatty acids, and a mixture thereof. Nonlimiting examples of fatty acid derivatives include ricinoleic acid, glycerol monostearate, 12-hydroxy stearic acid, ethyl stearate, cetyl stearate, cetyl palmitate, polyoxyethylene cetyl ether stearate, polyoxyethylene stearyl ether stearate, polyoxyethylene lauryl ether stearate, ethyleneglycol monostearate, polyoxyethylene monostearate, polyoxyethylene distearate, propyleneglycol monostearate, propyleneglycol distearate, trimethylolpropane distearate, sorbitan stearate, polyglyceryl stearate, dimethyl sebacate, PEG-15 cocoate, PPG-15 stearate, glyceryl monostearate, glyceryl distearate, glyceryl tristearate, PEG-8 laurate, PPG-2 isostearate, PPG-9 laurate, and a mixture thereof. Preferred for use herein are glycerol monostearate, 12-hydroxy stearic acid, and a mixture thereof.

In some cases, the one or more fatty compounds may be one or more high melting point fatty compounds. A high melting point fatty compound is a fatty compound having a melting point of 25° C. Even higher melting point fatty compounds may also be used, for example, fatty compounds having a melting point of 40° C. or higher, 45° C. or higher, 50° C. or higher. The high melting point fatty compound may be selected from the group consisting of fatty acids, fatty alcohol derivatives, fatty acid derivatives, and mixtures thereof. Nonlimiting examples of the high melting point compounds are found in the International Cosmetic Ingredient Dictionary, Sixteenth Edition, 2016, which is incorporated by reference herein in its entirety.

In some instances, fatty compounds include one or more waxes. The waxes generally have a melting point of from 35-120° C., at atmospheric pressure. Non-limiting examples of waxes in this category include for example, synthetic wax, ceresin, paraffin, ozokerite, illipe butter, beeswax, carnauba, microcrystalline, lanolin, lanolin derivatives, candelilla, cocoa butter, shellac wax, spermaceti, bran wax, capok wax, sugar cane wax, montan wax, whale wax, bayberry wax, sunflower seed wax (Helianthus annuus), acacia decurrents flower wax, or a mixture thereof.

Mention may be made, among the waxes capable of being used as non-silicone fatty compounds, of animal waxes, such as beeswax; vegetable waxes, such as sunflower seed (Helianthus annuus), carnauba, candelilla, ouricury or japan wax or cork fibre or sugarcane waxes; mineral waxes, for example paraffin or lignite wax or microcrystalline waxes or ozokerites; synthetic waxes, including polyethylene waxes, and waxes obtained by the Fischer-Tropsch synthesis.

In some instance, the fatty compounds include one or more non-silicone oils. The oils include, but are not limited to, natural oils, such as coconut oil; hydrocarbons, such as mineral oil and hydrogenated polyisobutene; fatty alcohols, such as octyldodecanol; esters, such as C₁₂-C₁₅ alkyl benzoate; diesters, such as propylene dipelarganate; and triesters, such as glyceryl trioctanoate. Suitable low viscosity oils have a viscosity of 5-100 mPas at 25° C., and are generally esters having the structure RCO—OR′ wherein RCO represents the carboxylic acid radical and wherein OR′ is an alcohol residue. Examples of these low viscosity oils include isotridecyl isononanoate, PEG-4 diheptanoate, isostearyl neopentanoate, tridecyl neopentanoate, cetyl octanoate, cetyl palmitate, cetyl ricinoleate, cetyl stearate, cetyl myristate, coco-dicaprylate/caprate, decyl isostearate, isodecyl oleate, isodecyl neopentanoate, isohexyl neopentanoate, octyl palmitate, dioctyl malate, tridecyl octanoate, myristyl myristate, octododecanol, or combinations of octyldodecanol, acetylated lanolin alcohol, cetyl acetate, isododecanol, polyglyceryl-3-diisostearate, or combinations thereof. The high viscosity oils generally have a viscosity of 200-1,000,000, or 100,000-250,000, mPas at 25° C. Such oils include castor oil, lanolin and lanolin derivatives, triisocetyl citrate, sorbitan sesquioleate, C₁₀-C₁₈ triglycerides, caprylic/capric/triglycerides, coconut oil, corn oil, cottonseed oil, glyceryl triacetyl hydroxystearate, glyceryl triacetyl ricinoleate, glyceryl trioctanoate, hydrogenated castor oil, linseed oil, mink oil, olive oil, palm oil, illipe butter, rapeseed oil, soybean oil, sunflower seed oil, tallow, tricaprin, trihydroxystearin, triisostearin, trilaurin, trilinolein, trimyristin, triolein, tripalmitin, tristearin, walnut oil, wheat germ oil, cholesterol, or combinations thereof.

Mineral oils, such as liquid paraffin or liquid petroleum, or animal oils, such as perhydrosqualene or arara oil, or alternatively of vegetable oils, such as sweet almond, calophyllum, palm, castor, avocado, jojoba, olive or cereal germ oil, may be utilized. It is also possible to use esters of these oils, e.g., jojoba esters. Also useful are esters of lanolic acid, of oleic acid, of lauric acid, of stearic acid or of myristic acid; esters of alcohols, such as oleyl alcohol, linoleyl or linolenyl alcohol, isostearyl alcohol or octyldodecanol; and/or acetylglycerides, octanoates, decanoates or ricinoleates of alcohols or of polyalcohols. It is alternatively possible to use hydrogenated oils which are solid at 25° C., such as hydrogenated castor, palm or coconut oils, or hydrogenated tallow; mono-, di-, tri- or sucroglycerides; lanolins; or fatty esters which are solid at 25° C.

### EXAMPLES

Implementation of the present disclosure is provided by way of the following examples. The examples serve to illustrate the technology without being limiting in nature.

### Example 1

(Inventive & Comparative Compositions)

| | | | | Inventive | | | | Comparative | | | | |
|---|---|---|---|---|---|---|---|---|---|---|---|---|
| | | | | A | B | C | D | E | F | G | H | I |
| (a) | Halogenated | Xanthene Dye | D&C RED 28 | 0.02 | 0.02 | | | | | | | |
| | Direct Dye | Triarylmethane | TETRABROMOPHENOL | | 0.012 | | | | | | | |
| | | Dye | BLUE | | | | | | | | | |
| | | | TETRABROMOPHENOL | | | 0.02 | | | | | | |
| | | | BLUE | | | | | | | | | |
| | | Azo Dye | BASIC RED 18 | | | | 0.02 | | | | | |
| | Non- | Xanthene Dye | D&C YELLOW 8/Fluorescein | | | | | 0.02 | | | | |
| | Halogenated | Triarylmethane | M-CRESOL- | | | | | | 0.02 | | | |
| | Direct Dye | Dye | SULFONEPHTHALEIN | | | | | | | | | |
| | | Azo Dye | ACID ORANGE 8 | | | | | | | 0.02 | | |
| | | Thiazine Dye | BASIC GREEN 5 | | | | | | | | 0.02 | |
| | | Nitro Dye | ACID YELLOW 24 | | | | | | | | | 0.02 |
| (b) | Hydrogen Peroxide | | | 11.8 | 11.8 | 11.8 | 11.8 | 11.8 | 11.8 | 11.8 | 11.8 | 11.8 |
| (d) | Fatty Alcohol | | CETEARYL ALCOHOL | 2.3 | 2.3 | 2.3 | 2.3 | 2.3 | 2.3 | 2.3 | 2.3 | 2.3 |
| (e) | Nonionic Surfactant | | CETEARETH-25, | 1.8 | 1.8 | 1.8 | 1.8 | 1.8 | 1.8 | 1.8 | 1.8 | 1.8 |
| | | | STEARETH-20, AND PEG-4 | | | | | | | | | |
| | | | RAPESEEDAMIDE | | | | | | | | | |
| (f) | Water Soluble Solvent | | GLYCERIN | 0.5 | 0.5 | 0.5 | 0.5 | 0.5 | 0.5 | 0.5 | 0.5 | 0.5 |
| | Fragrance, Preservatives, Chelating Agents, Inorganic Salts, | | | ≤3 | ≤3 | ≤3 | ≤3 | ≤3 | ≤3 | ≤3 | ≤3 | ≤3 |
| | pH Adjuster, Botanical Extracts, Vitamins, etc | | | | | | | | | | | |
| (c) | Water | | WATER | QS | QS | QS | QS | QS | QS | QS | QS | QS |
| | | | | 100 | 100 | 100 | 100 | 100 | 100 | 100 | 100 | 100 |

### Example 2

### Inventive Emulsions

| | | | J | K | L |
|---|---|---|---|---|---|
| (a) | Halogenated | Tetrabromophenol | 0.02 | — | — |
| | | Blue | | | |
| | Direct Dye | Red 28 | — | 0.02 | — |
| | | HC Yellow No. 16 | — | — | 0.02 |
| (b) | Hydrogen | Hydrogen Peroxide | 18 | 18 | 18 |
| | Peroxide | | | | |
| (d) | Fatty Alcohol | Cetearyl Alcohol | 3.15 | 3.15 | 3.15 |
| (e) | Nonionic | PEG-40 Hydrogenated | 0.9 | 0.9 | 0.9 |
| | Surfactant | Castor Oil | | | |
| | Anionic | Sodium Cetearyl | 0.45 | 0.45 | 0.45 |
| | Surfactant | Sulfate | | | |
| | Fragrance, Preservatives, | | ≤3 | ≤3 | ≤3 |
| | Chelating Agents, Inorganic | | | | |
| | Salts, pH Adjuster, Botanical | | | | |
| | Extracts, Vitamins, etc | | | | |
| (f) | Fatty | Mineral Oil | 17 | 17 | 17 |
| | Compound | | | | |
| | (oil) | | | | |
| (c) | | Water | 59.98 | 59.98 | 59.98 |

The foregoing description illustrates and describes the invention. The disclosure shows and describes only the preferred embodiments but it should be understood that the invention is capable to use in various other combinations, modifications, and environments and is capable of changes or modifications within the scope of the inventive concepts as expressed herein, commensurate with the above teachings and/or the skill or knowledge of the relevant art. The embodiments described herein above are further intended to explain best modes known by applicant and to enable others skilled in the art to utilize the disclosure in such, or other, embodiments and with the various modifications required by the particular applications or uses thereof. Accordingly, the description is not intended to limit the invention to the form disclosed herein.

The term “hair” as used herein includes hair of the head, the face (including beard hair and mustache hair), eyebrows, eyelashes, and body hair, unless otherwise specified.

As used herein, the terms “comprising,” “having,” and “including” are used in their open, non-limiting sense.

A “developer composition” as used herein is a composition containing one or more oxidizing agents, preferably a peroxide (hydrogen peroxide) and is mixed with a hair coloring base composition to form a ready-to-use hair coloring composition.

A “hair coloring base composition” as used herein is a hair coloring composition containing one or more oxidative dye precursors and is mixed with a developer composition to form a ready-to-use hair coloring composition.

A “ready-to-use hair coloring composition” is an “active” composition that includes one or more oxidative dye precursors and one or more oxidizing agents; and is formed by combining a hair coloring base composition with a developer composition.

A “composition colorant” is a compound that colors the composition but does not have an appreciable coloring effect on hair. In other words, the composition colorant is included to provide color to the composition, for example, for aesthetic appeal. It is not included to impart color to the hair. Styling gels, for example, can be found in a variety of different colors (e.g., light blue, light pink, etc.) yet application of the styling gel to the hair does not change the color of the hair.

The terms “a,” “an,” and “the” are understood to encompass the plural as well as the singular. Thus, the term “a mixture thereof” also relates to “mixtures thereof.” Throughout the disclosure, the term “a mixture thereof” is used, following a list of elements as shown in the following example where letters A-F represent the elements: “one or more elements selected from the group consisting of A, B, C, D, E, F, and a mixture thereof.” The term, “a mixture thereof” does not require that the mixture include all of A, B, C, D, E, and F (although all of A, B, C, D, E, and F may be included). Rather, it indicates that a mixture of any two or more of A, B, C, D, E, and F can be included. In other words, it is equivalent to the phrase “one or more elements selected from the group consisting of A, B, C, D, E, F, and a mixture of any two or more of A, B, C, D, E, and F.”

Likewise, the term “a salt thereof” also relates to “salts thereof.” Thus, where the disclosure refers to “an element selected from the group consisting of A, B, C, D, E, F, a salt thereof, and a mixture thereof,” it indicates that that one or more of A, B, C, D, and F may be included, one or more of a salt of A, a salt of B, a salt of C, a salt of D, a salt of E, and a salt of F may be include, or a mixture of any two of A, B, C, D, E, F, a salt of A, a salt of B, a salt of C, a salt of D, a salt of E, and a salt of F may be included.

The expression “one or more” means “at least one” and thus includes individual components as well as mixtures/combinations.

Other than in the operating examples, or where otherwise indicated, all numbers expressing quantities of ingredients and/or reaction conditions may be modified in all instances by the term “about,” meaning within +/−5% of the indicated number.

Some of the various categories of components identified may overlap. In such cases where overlap may exist and the composition includes both components (or the composition includes more than two components that overlap), an overlapping compound does not represent more than one component. For example, a nonionic surfactant may be considered both a nonionic surfactant and a fatty compound. If a particular composition includes both a nonionic surfactant and a fatty compound, a single compound will serve as only the nonionic surfactant or the fatty compound (the single compound does not serve as both the nonionic surfactant and the fatty component).

The salts referred to throughout the disclosure may include salts having a counter-ion such as an alkali metal, alkaline earth metal, or ammonium counter-ion. This list of counter-ions, however, is non-limiting.

The term “treat” (and its grammatical variations) as used herein refers to the application of the compositions of the present disclosure onto a keratinous substrate such as hair. The term ‘treat” (and its grammatical variations) as used herein also refers to contacting keratinous substrates such as hair with the compositions of the present disclosure.

As used herein, all ranges provided are meant to include every specific range within, and combination of sub ranges between, the given ranges. Thus, a range from 1-5, includes specifically 1, 2, 3, 4 and 5, as well as sub ranges such as 2-5, 3-5, 2-3, 2-4, 1-4, etc. All ranges and values disclosed herein are inclusive and combinable. For examples, any value or point described herein that falls within a range described herein can serve as a minimum or maximum value to derive a sub-range, etc.

All components positively set forth throughout the instant disclosure may be negatively excluded from the claims, e.g., a claimed composition may be “free,” “essentially free” (or “substantially free”) of one or more components that are positively set forth in the instant disclosure. As an example, silicones can optionally be included in the compositions but preferably the compositions are free or essentially free from silicones. Silicones are synthetic polymers made up of repeating units of siloxane, elemental silicon and oxygen, combined with other elements, most often carbon and hydrogen. Thus, silicones are also called polysiloxanes.

The term “substantially free” or “essentially free” as used herein means that there is less than about 2% by weight of a specific material added to a composition, based on the total weight of the compositions. Nonetheless, the compositions may include less than about 1 wt. %, less than about 0.5 wt. %, less than about 0.1 wt. %, or none of the specified material. All of the components set forth herein may be optionally included or excluded from the compositions/method/kits. When excluded, the compositions/methods/kits may be free or essentially free of the component. For example, a particular composition may be free or essentially free of alkoxylated compounds, for example, ethoxylated thickeners and/or ethoxylated surfactants. Likewise, a particular composition may be free or essentially free of sulfates, such as sulfate surfactants.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

1. A developer composition comprising:
(a) about 0.001 to about 5 wt. % of one or more direct dyes having at least one halogenated aromatic ring;
(b) about 1 to about 40 wt. % of one or more oxidizing agents; and
(c) about 50 to 90 wt. % of water;
wherein the composition has a pH from about 1 to about 4, and
all weight percentages are based on the total weight of the composition.

2. The developer composition of claim 1, wherein the one or more direct dyes are chosen from triarylmethane dyes, xanthene dyes, azo dyes, and mixtures thereof.

3. The developer composition of claim 2 comprising one or more xanthene dyes chosen from D&C Red 28, Erythrosine B, Eosin Y, Eosin B, (eosin (2′,4′,5′,7′-tetrabromo-fluorescein) methyl ester, eosin (2′,4′,5′,7′-tetrabromo-fluorescein) p-isopropylbenzyl ester, eosin derivative (2′,7′-dibromo-fluorescein), eosin derivative (4′,5′-dibromo-fluorescein), eosin derivative (2′,7′-dichloro-fluorescein), eosin derivative (4′,5′-dichloro-fluorescein), eosin derivative (2′,7′-diiodo-fluorescein), eosin derivative (4′,5′-diiodo-fluorescein), eosin derivative (tribromo-fluorescein), eosin derivative (2′,4′,5′,7′-tetrachloro-fluorescein), erythrosin B (2′,4′,5′,7′-tetraiodo-fluorescein), erythiosin B; fluorescein; fluorescein dianion, phloxin B (2′,4′,5′,7′-tetrabromo-3,4,5,6-tetrachloro-fluorescein, dianion), phloxin B (tetrachloro-tetrabromo-fluorescein), phloxine B, rose bengal (3,4,5,6-tetrachloro-2′,4′,5′,7′-tetraiodofluorescein, dianion), pyronin G, pyronin J, pyronin Y, 4,5-dibromo-rhodamine methyl ester, 4,5-dibromo-rhodamine n-butyl ester, rhodamine 101 methyl ester, rhodamine 123, rhodamine 6G, rhodamine 6G hexyl ester, tetrabromo-rhodamine 123, tetramethyl-rhodamine ethyl ester, and mixtures thereof.

4. The developer composition of claim 3 comprising D&C red 29.

5. The developer composition of claim 2 comprising one or more triarylmethane dyes chosen from tetrabromophenol blue, 3,4,5,6-tribromophenol sulfone phthalein, sulfobromophthalein, bromocresol green, bromothymol blue, and mixtures thereof.

6. The developer composition of claim 5 comprising tetrabromophenol blue.

7. The developer composition of claim 2 comprising one or more azo dyes chosen from Acid Red 337, Disperse Red 167, Basic Red 18, Disperse Red 118, HC Yellow 16, HC Blue 18, 2-chloro-4-[(E)-(3-methyl-1,2,4-thiadiazol-5-yl)diazenyl]phenol, and mixtures thereof.

8. The developer composition of any one of the above claims, wherein the one or more oxidizing agents are chosen from hydrogen peroxide, inorganic alkali metal peroxides, organic peroxides, inorganic perhydrate salts, bromates, and a mixture thereof.

9. The developer composition of claim 8 comprising hydrogen peroxide.

10. The developer composition of claim 1, further comprising:
(d) one or more fatty alcohols.

11. The developer composition of claim 10, wherein the composition is in the form of an emulsion, further comprising about 5 to about 40 wt. % of one or more oils other than the one or more fatty alcohols of (d).

12. The developer composition of claim 1, further comprising:
(e) one or more nonionic surfactants.

13. The developer composition of claim 1, further comprising:
(f) one or more water-soluble organic solvents.

14. The developer composition of claim 1, further comprising:
(g) about 0.01 to about 6 wt. % of one or more miscellaneous ingredients chosen from fragrances, preservatives, chelating agents, pH adjusters, buffering agents, botanical extracts, and a mixture thereof.

15. The developer composition of claim 1, wherein
the pH of the compositions does not change by more than 15% after 1 month in storage at 25° C.; or
at least 90% of peroxide content remains after 1 month in storage at 25° C.

16. The developer composition of claim 1 being free from oxidative dye precursors.

17. A developer compositions composition comprising:
(a) about 0.005 to about 2 wt. % of one or more direct dyes having at least one halogenated aromatic ring;
(b) about 1 to about 40 wt. % of one or more oxidizing agents chosen from hydrogen peroxide, inorganic alkali metal peroxides, organic peroxides, inorganic perhydrate salts, bromates, and a mixture thereof;
(c) about 70 to 90 wt. % of water;
(d) about 0.5 to about 10 wt. % of one or more fatty alcohols;
(e) about 0.1 to about 10 wt. % of one or more nonionic surfactants; and
(f) optionally, about 0.1 to about 30 wt. % of one or more water-soluble solvents;
(g) optionally, about 0.01 to about 6 wt. % one or more miscellaneous ingredients chosen from fragrances, preservatives, chelating agents, pH adjusters, buffering agents, botanical extracts, and a mixture thereof;
wherein the developer composition has a pH of about 1 to about 4, and
all weight percentages are based on the total weight of the composition.

18. The developer composition of claim 17 comprising:
(a) about 0.005 to about 2 wt. % of one or more direct dyes comprising having at least one halogenated aromatic ring chosen from triarylmethane dyes, xanthene dyes, azo dyes, and mixtures thereof;
(b) about 1 to about 40 wt. % of hydrogen peroxide;
(c) about 70 to 90 wt. % of water;
(d) about 0.5 to about 10 wt. % of one or more fatty alcohols;
(e) about 0.1 to about 10 wt. % of one or more nonionic surfactants;
(f) about 0.1 to about 30 wt. % of one or more water-soluble solvents chosen from glycerin, mono-alcohols, polyols (polyhydric alcohols), glycols, and a mixture thereof;
(g) about 0.01 to about 4 wt. % of one or more miscellaneous ingredients chosen from fragrances, preservatives, chelating agents, pH adjusters, buffering agents, botanical extracts, and a mixture thereof.

19. A method for coloring hair comprising:
(i) mixing the developer composition of claim 1 with a hair coloring base composition comprising one or more oxidative dye precursors to form an active hair coloring composition;
(ii) applying the active hair coloring compositions to the hair; and
(iii) after applying the active hair coloring composition to the hair, allowing the active hair coloring composition to remain on the hair for a period of time; and
(iv) after the period of time, rinsing the active hair coloring composition from the hair.

20. A kit comprising:
(i) the developer composition of claim 1; and
(ii) a hair coloring base composition comprising one or more oxidative dye precursors and one or more reducing agents;
wherein the developer composition and the hair coloring base composition are separately contained.
